(19) United States
(12) Patent Application Publication
(10) Pub. No.: US 2025/0302530 A1
(43) Pub. Date: Oct. 2, 2025

(54) APPARATUS AND METHOD FOR THE LASER TREATMENT OF GASTROESOPHAGEAL REFLUX DISEASE

(71) Applicant: Tecure, Inc., Busan (KR)

(72) Inventors: Hye Jin KIM, Busan (KR); Van Gia TRUONG, Busan (KR)

(21) Appl. No.: 18/739,608

(22) Filed: Jun. 11, 2024

(30) Foreign Application Priority Data
Mar. 29, 2024 (KR) 1020240042908

(51) Int. Cl. A61B 18/24; A61B 18/00

(52) U.S. Cl. CPC A61B 18/24; A61B 2018/00023; A61B 2018/0022; A61B 2018/00488; A61B 2018/00696; A61B 2018/00791; A61B 2018/00875; A61B 2018/00904

(57) ABSTRACT

The present disclosure relates to an apparatus and a method for a laser treatment of gastro-esophageal reflux disease using a balloon catheter.

### CROSS-REFERENCE TO RELATED APPLICATION(S)

This application claims benefit of priority to Korean Patent Application No. 10-2024-0042908 filed on Mar. 29, 2024, in the Korean Intellectual Property Office, the disclosure of which is incorporated herein by reference in its entirety.

### BACKGROUND

### 1. Field

The present disclosure relates to an apparatus and a method for a laser treatment of gastro-esophageal reflux disease using a balloon catheter, and particularly relates to an apparatus and a method for laser treatment of gastro-esophageal reflux disease using a balloon catheter in which in reflux disease caused by sphincter dysfunction or esophageal precancerous lesions, by selectively treating a sphincter layer or a mucous membrane with photothermal treatment without damaging surrounding tissue, a diameter of a sphincter may be reduced by increasing a size of a smooth muscle, and by cauterizing a precancerous tissue in the mucous membrane, cells and tissues may be rearranged.

### 2. Description of Related Art

Gastro-Esophageal Reflux Disease (GERD) is a common digestive disorder with an estimated incidence of 18 to 25% in North America and has been gradually increasing in recent decades. GERD occurs when stomach acid refluxes into the esophagus due to a weak or relaxed Lower Esophageal Sphincter (LES). The LES is a junction between the esophagus and the stomach, known as a muscular ring, and comprises a smooth muscle layer and maintains tonic contraction by muscle and nerve factors.

Typically, patients with GERD experience major symptoms such as chest pain, heartburn discomfort, and dysphagia. When GERD is left untreated, complications may include esophageal ulcers, esophageal strictures, and esophageal erosion. A variety of medical and surgical treatments have been developed to treat the GERD indirectly or directly.

Although endoscopic therapy has evolved into a potentially safe and effective treatment option for GERD, FDA-approved products for the GERD market include a Medigus ultrasonic surgical endostapler (MUSETM, Medigus, Omer, Israel), transoral incisionless fundus surgery (EsophyX, EndoGastric Solution, WA, USA), and Stretta therapy (Restech, Houston, TX, USA). Medigus Ultrasound Surgical Endoscopic Surgery is an intraoral incision-free anterior fundus augmentation using an ultrasound-integrated surgical stapler under endoscopic guidance. Stretta therapy applies RF energy to remodel the sphincter through direct contact of multiple electrodes in a muscle layer of the LES. A diameter of the LES may be reduced to reduce a frequency of reflux of stomach contents into the esophagus. However, the efficacy of these treatment methods is often unknown and these treatment methods may be associated with lack of normalization of esophageal acid exposure in numerous patients and limited effectiveness in healing.

Additionally, these treatment methods require advanced technology and time-consuming surgery, the risk of procedural complications, such as dysphagia, chest pain, sore throat, bleeding, and perforation, increases.

The present disclosure may be applied not only to GERD but also to other gastro-esophageal reflux diseases. Barrett's Esophagus (BE) may be caused as a chronic complication of GERD, and is a precancerous lesion in which the risk of developing cancer increases as a period of reflux symptoms increases in a state in which a mucous membrane of a distal portion of the esophagus connected to the stomach is exposed to stomach acid for a long time due to continuous reflux to change esophageal mucosa tissue to gastric mucosa tissue. In the beginning, there are no special symptoms, but when complications such as ulcers or adenocarcinoma occur, this causes symptoms such as vomiting, difficulty swallowing, and vomiting blood. An incidence rate may increase due to obesity and aging, and the risk increases when there is a structural abnormality, such as a hiatal hernia.

Recently, endoscopic treatments such as radiofrequency ablation and cryoablation, rather than existing surgical methods, have become a hot topic for the treatment of BE. The Radiofrequency Ablation (RFA) is a procedure that uses high-frequency energy to generate heat through an electrode to cause damage to the epithelium of Barrett's esophagus and remove the same. Cryoablation is a procedure that rapidly cools and removes the epithelium using liquid nitrogen. However, these procedures have difficulty inducing unbalanced treatment effects and necrosis of an entire region, take a long time and may cause discomfort to the patient due to the risks of perforation, chest pain, esophageal stricture, and bleeding.

Accordingly, additional improvements are still required to ensure the effectiveness, costs, and treatment safety of endoscopic treatment devices for the treatment of gastro-esophageal reflux diseases.
(Patent Document 1) Korean Patent No. 10-1971185 (Apr. 16, 2019)

### SUMMARY

In order to resolve the above-described problems, an aspect of the present disclosure is to provide an apparatus and a method for a laser treatment of a gastro-esophageal reflux disease using a balloon catheter in which in a sphincter dysfunction, a size of the smooth muscle may increase to reduce a diameter of the sphincter using a photothermal treatment in a sphincter layer without damaging surrounding tissues, thereby rearranging the esophageal mucosal tissue, or Barrett's esophagus in which esophageal mucosal tissue is transformed into stomach tissue may be regenerated into a normal esophageal mucosal tissue by cauterizing precancerous mucosal tissue using the photothermal treatment in the mucosal layer without damaging the surrounding tissues.

Additionally, an aspect of the present disclosure is to treat precise areas with various balloon shapes.

Additionally, an aspect of the present disclosure is to treat an area to be treated without moving a balloon.

In order to achieve the above-describe objects, the present disclosure provides an apparatus and a method for a laser treatment of gastro-esophageal reflux disease using a balloon catheter is as follows.

An apparatus for laser treatment of a gastro-esophageal reflux disease using a balloon catheter according to an example embodiment may include: a light irradiation unit configured to irradiate light to tissue inside the esophagus; a balloon catheter including a first balloon portion into which an optical fiber connected to the light irradiation unit is inserted, and which is expanded by fluid to expand the esophageal tissue and is expanded to form a straight line having an angle of 0 to 90° with a movement path of the optical fiber, and a second balloon portion expanded to form a curved line; a sensor monitoring unit configured to monitor esophageal tissue sensed by a sensor provided in the balloon catheter; and a controller configured to receive measurement information from the sensor monitoring unit to control a light source of the light irradiation unit.

Additionally, the apparatus for laser treatment of gastro-esophageal reflux disease using a balloon catheter may further include: a fluid management unit configured to supply fluid to expand the balloon catheter and remove the supplied fluid, wherein the fluid management unit is controlled by the controller.

Additionally, the first balloon portion and the second balloon portion may be expanded into a region rotated around a movement path of the light irradiation unit.

Additionally, the first balloon portion and the second balloon portion may be disposed to overlap each other in at least a partial region in a movement direction of the optical fiber.

Additionally, the first balloon portion and the second balloon portion may be supplied with fluid separately by the controller.

Additionally, a plurality of the sensors may form an array and may be provided on an outer side of the first balloon portion.

Additionally, the fluid management unit may include an air trap remover configured to remove an air trap inside the balloon catheter, and a cooler configured to generate a cooling fluid for lowering a temperature of the fluid.

Additionally, the balloon catheter may include: the optical fiber; an optical fiber tip formed to surround the inserted optical fiber and configured to uniformly distribute light emitted from the optical fiber to the esophageal tissue; a balloon formed of a transparent material so that light emitted from the optical fiber tip is irradiated to the esophageal tissue and expanded when fluid flows therein, and including the first balloon portion and the second balloon portion; a flexible tip disposed in a front end to be guided and inserted into the esophageal tissue; and a guide wire configured to guide by penetrating through the flexible tip.

Additionally, the sensor array may measure a temperature, tissue deformation, pH, and a mucosal impedance of the esophageal tissue.

Additionally, the apparatus for laser treatment of gastro-esophageal reflux disease using a balloon catheter may further include: a delivery tubing provided with a first channel for accommodating a sensor wire connected to the sensor, a second channel for accommodating the optical fiber and allowing for entry and exit of a material to expand the balloon catheter, and a third channel for accommodating the guide wire to secure an entry path; and an endoscope portion formed to surround at least a portion of the delivery tubing.

Additionally, the endoscope portion may include: an endoscope camera; and a filter attached to the endoscope portion and configured to protect the endoscope camera.

Additionally, the cooling fluid may be injected to flow along an inner surface of the balloon or the movement path.

Additionally, when a length covered by the first balloon portion in a direction perpendicular to the movement path is defined as a first radius, the first radius may be 7.5 to 15 mm, and when a length covered by the second balloon portion in the direction perpendicular to the movement path is defined as a second diameter, the second diameter may be greater than the first radius and less than 40 mm.

Additionally, an angle between any tangent line in contact with the second balloon portion and the movement path is 20 to 60°.

Additionally, the apparatus for laser treatment of gastro-esophageal reflux disease using a balloon catheter may further include: a reflecting mirror configured to reflect light generated from the light irradiation unit and disposed inside the balloon catheter.

Additionally, the second balloon portion is provided in plural.

An apparatus for laser treatment of gastro-esophageal reflux disease using a balloon catheter according to another example embodiment of the present disclosure may include: a light irradiation unit configured to irradiate light to esophageal tissue; a balloon catheter having the light irradiation unit inserted thereinto, expanded by fluid so that the light irradiation unit is disposed on one side of an interior, and configured to expand the esophageal tissue; a sensor monitoring unit configured to monitor the esophageal tissue sensed by a sensor provided in the balloon catheter; and a controller configured to receive measurement information from the sensor monitoring unit to control a light source of the light irradiation unit.

Additionally, the light irradiation unit and the balloon catheter may be provided in plural, and the controller may individually control the light irradiation unit and the balloon catheter.

Additionally, the apparatus for laser treatment of gastro-esophageal reflux disease using a balloon catheter may be provided with an optical mask configured to cover light emitted from an optical fiber tip disposed in an end of the optical fiber.

A method for a laser treatment of gastro-esophageal reflux disease using a balloon catheter according to an example embodiment of the present disclosure may include an insertion operation of inserting a balloon catheter through an endoscope channel; an inflating operation of injecting fluid into the balloon catheter and disposing the fluid on esophageal tissue; an adjustment operation of adjusting an amount of fluid to be adjusted to an irradiation position through a sensor provided on an outer side of the balloon catheter; an irradiation operation of irradiating light to the irradiation position; and a moving operation of moving a position of an optical fiber inside the balloon catheter and irradiating light.

In the present disclosure, in gastro-esophageal reflux diseases, a size of the smooth muscle may increase to reduce a diameter of the sphincter using a photothermal treatment in a sphincter layer without damaging surrounding tissues, or precancerous mucosal tissue may be cauterized using the photothermal treatment on a mucosal layer, thereby rearranging an esophageal mucosal tissue.

### BRIEF DESCRIPTION OF DRAWINGS

The above and other aspects, features, and advantages of the present disclosure will be more clearly understood from the following detailed description, taken in conjunction with the accompanying drawings, in which:

FIG. 1 is a schematic diagram illustrating an overall configuration of an apparatus for laser treatment of gastro-esophageal reflux disease using a balloon catheter according to an example embodiment of the present disclosure;

FIGS. 2 and 3 illustrate a state in which fluid is injected into an apparatus for laser treatment using a balloon catheter, and FIG. 2 illustrates a concept of fluid movement through unidirectional fluid injection, and FIG. 3 illustrates a concept of fluid movement through fluid injection and discharge;

FIG. 4 and FIG. 5 are views illustrating a transmission tubing having a dual channel. FIG. 4 is a perspective view, and FIG. 5 is a cross-sectional view;

FIG. 6 and FIG. 7 illustrate a transmission tubing having a triple channel. FIG. 6 is a perspective view, and FIG. 7 is a cross-sectional view;

FIG. 8, FIG. 9 and FIG. 10 illustrate various structures of a flexible tip having a round end. FIG. 8 illustrates a shape of a flexible tip connected to a proximal portion of a balloon, and FIGS. 9 and 10 illustrate a shape of a round tip according to the presence or absence of insertion of a guide wire;

FIG. 11, FIG. 12, FIG. 13, and FIG. 14 illustrate flexible tips having a step portion. FIG. 11 illustrates a shape of the flexible tip having the step portion, connected to the balloon, and FIGS. 12, 13 and 14 illustrate various shapes of the flexible tip having the step portion for inserting a guide wire;

FIG. 15 illustrates a shape of the balloon according to a first embodiment;

FIGS. 16 and 17 are auxiliary views illustrating a shape of a balloon according to a first embodiment, and FIG. 16 is a case of having a first balloon portion in parallel with a movement direction, and FIG. 17 is a case of having a first balloon portion having an angle formed therein;

FIG. 18 is a schematic diagram of a first embodiment of an optical fiber tip connected to an optical fiber;

FIG. 19, FIG. 20, and FIG. 21 illustrate cases in which a conical diffusion tip for diffusion is further formed on an optical fiber tip;

FIG. 22, FIG. 23, and FIG. 24 are graphs illustrating an amount of light intensity change according to a position when the optical fiber tip of FIG. 19, FIG. 20, and FIG. 21 are used, and FIG. 19 is illustrated as FIG. 22, FIG. 20 is illustrated as FIG. 23, and FIG. 21 is illustrated as FIG. 24;

FIG. 25 illustrates a relationship between a balloon diameter and a fluid injection amount, FIG. 26 illustrates stress/strain rate and balloon pressure, and FIG. 27 is a graph illustrating a temperature change during treatment over time;

FIG. 28 is a flowchart of a treatment method using an apparatus for laser treatment using a balloon catheter according to an example embodiment of the present disclosure;

FIG. 29 and FIG. 30 schematically illustrate treatment of a plurality of treatment sites without moving a position of the balloon catheter in the human body. FIG. 29 illustrates primary treatment of a first site P1, and FIG. 30 illustrates secondary treatment of a second site P2;

FIG. 31, FIG. 32, FIG. 33, and FIG. 34 are cross-sectional views of tubular tissue after laser light treatment according to the present disclosure;

FIG. 35, FIG. 36, FIG. 37, and FIG. 38 are cross-sectional views of tubular tissue illustrating an effect after a cooling process after laser light treatment according to the present disclosure;

FIG. 39 and FIG. 40 illustrate an apparatus for laser treatment using a balloon catheter using a balloon catheter having a balloon shape according to a second embodiment.

FIG. 39 and FIG. 40 are cross-sectional views illustrating cross-sections, perpendicular to each other;

FIG. 41 is a schematic view of an apparatus for laser treatment using a balloon catheter having a balloon according to a third embodiment of the present disclosure;

FIG. 42, FIG. 43, FIG. 44 and FIG. 45 schematically illustrate the use of an apparatus for laser treatment using a balloon catheter having a balloon of the second embodiment or the third embodiment. FIG. 42 is a cross-sectional view illustrating a case in which an apparatus for laser treatment using a balloon catheter having a balloon of the second embodiment is disposed in a tissue, and FIGS. 43 and 44 illustrate a case in which a second balloon portion is inflated in the apparatus for laser treatment using a balloon catheter having a balloon of the third embodiment. FIG. 45 illustrates a state in which the laser treatment is performed on a mucous membrane and a submucosal layer;

FIG. 46, FIG. 47 and FIG. 48 schematically illustrate the use of an apparatus for laser treatment using a balloon catheter having a balloon according to the third embodiment, and in the case of having two second balloon portions, FIGS. 46 and 47 illustrate a case in which sizes of the second balloon portions are the same, and FIG. 48 illustrate a case in which the sizes of the second balloon are different;

FIG. 49, FIG. 50 and FIG. 51 illustrate cases in which a plurality of balloon catheters are provided in an apparatus for laser treatment using a balloon catheter having a balloon according to the second embodiment. FIG. 49 is a case in which the balloon is expanded to the same size and light is irradiated therein, FIG. 50 is a case in which light is not irradiated from an interior of some balloon catheters, and FIG. 51 is a case in which sizes of the balloons are different;

FIG. 52 and FIG. 53 illustrate controlling an irradiation range of laser light by an optical mask in a balloon catheter having a balloon according to the second or third embodiment, and FIG. 52 illustrates a state not covered by an optical mask, and FIG. 53 illustrates a state partially covered by the optical mask;

FIG. 54 is a schematic diagram illustrating a state in which an apparatus for laser treatment using a balloon catheter having a balloon according to an example embodiment of the present disclosure is disposed in a tissue;

FIG. 55 is a schematic diagram illustrating a state in which an apparatus for laser treatment using a balloon catheter having a balloon according to an example embodiment of the present disclosure is disposed in a tissue;

FIG. 56 is a schematic diagram illustrating a state in which an apparatus for laser treatment using a balloon catheter having a balloon according to an example embodiment of the present disclosure is disposed in a tissue;

FIG. 57 and FIG. 58 are graphs illustrating effects on esophageal tissue before and after treatment through an apparatus or a method for a laser treatment using a balloon catheter according to an example embodiment of the present disclosure. FIG. 57 is a graph illustrating effects on esophageal acidity and muscle layer before and after treatment for an esophageal reflux disease according to an embodiment. FIG. 58 is a graph illustrating a change in electronic sensor sensitivity before and after treatment of Barrett's esophageal tissue according to another example embodiment of the present disclosure and an effective range comparison through endoscopic screening;

FIG. 59, FIG. 60 and FIG. 61 illustrate various shapes of an optical fiber tip according to an example embodiment of the present disclosure. FIG. 59 illustrates a diffusing fiber, FIG. 60 illustrates a radial fiber, and FIG. 61 illustrates a side-firing fiber; and

FIG. 62, FIG. 63 and FIG. 64 schematically illustrate the use of an apparatus for laser treatment using a balloon catheter. FIG. 59 and FIG. 60 are illustrated as FIG. 62, and FIG. 61 is illustrated as FIG. 63 to FIG. 64.

### DETAILED DESCRIPTION

Hereinafter, with reference to the drawings, specific embodiments of the present disclosure will be described. However, the concept of the present disclosure is not limited to the suggested embodiments, and those skilled in the art who understand the concept of the present disclosure may propose other embodiments included within the scope of the concept of other regressive disclosures or the present disclosure by adding, modifying, or deleting components, but the embodiments described herein will also be considered to be included within the scope of the present disclosure.

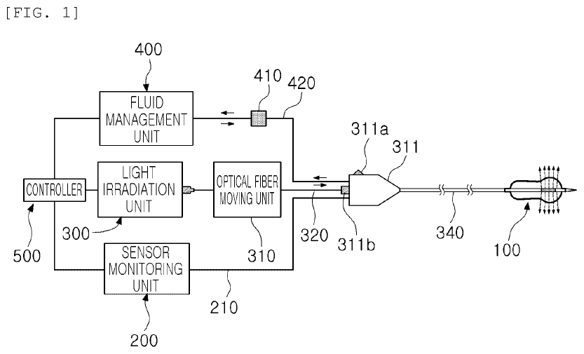

FIG. 1 is a schematic diagram illustrating an overall configuration of an apparatus for laser treatment of gastro-esophageal reflux disease using a balloon catheter according to an example embodiment of the present disclosure.

An apparatus for a sphincter light treatment according to an example embodiment of the present disclosure may include a balloon catheter 100, a sensor monitoring unit 200, a light irradiation unit 300, a fluid management unit 400, and a controller 500.

The light irradiation unit 300 includes a light source for irradiating light to a sphincter tissue.

For example, the light irradiation unit 300 irradiates light to esophageal tissue according to a movement of an optical fiber 320 inserted into the balloon catheter 100.

The balloon catheter 100 is provided with a flexible tip 130 in one end thereof and is guided and inserted into the esophageal tissue, and the balloon catheter 100 is expanded by fluid to expand tissue inside the esophagus.

When the balloon catheter 100 reaches a desired position in order to consistently expand an internal structure of a stenosis or narrowed tissue, the fluid may be injected into the balloon catheter 100. Additionally, the flexible tip 130 is provided to allow for an entry into the body while minimizing mechanical damage to body tissues.

The sensor monitoring unit 200 may receive information sensed from the sensor 221 provided in the balloon catheter 100. The sensor 221 may be provided in plural and a plurality of sensors 221 may form a sensor array 220. Physical parameters of the esophageal tissue sensed by the sensor 221 or the sensor array 220 are monitored.

An optical fiber 320 is provided inside the balloon catheter 100 to irradiate light to the esophageal tissue as needed.

For example, the balloon catheter 100 may include a balloon 150 (see FIG. 15) including a first balloon portion 151 (see FIG. 15) expanded by forming a straight line having an angle of 0 to 90° with a movement path or a movement direction of the optical fiber 320, and a second balloon portion 152 (see FIG. 15) connected to the first balloon portion 151 and expanded by forming a curved line.

The first balloon portion 151 and the second balloon portion 152 may be directly connected to each other, so that the fluid supplied inside may move inside the first balloon portion 151 and the second balloon portion 152, and the fluid supplied into the first balloon portion 151 and the second balloon portion 152 may not move.

For example, the balloon 150 included in the balloon catheter 100 may include silicone, polyurethane, nylon, elastomer, and other thermoplastic elastic materials. Additionally, depending on the material, the pressure formed inside may be 0.1 to 100 PSI. Specifically, at a pressure of 20 to 100 PSI, the material of the balloon 150 may be acrylic, polyethylene terephthalate (PET), and nylon, which may be expanded to a small expansion range of 0 to 10%. Additionally, in the case of the balloon 150 using silicone, polyurethane, nylon elastomer, and the like, the pressure from 0.1 to 5 PSI may be applied thereto and the balloon 150 may be expanded to a large expansion range of 10 to 200%. A shape of the expanded balloon 150 may be formed differently depending on the type of tubular tissue.

The controller 500 may receive measurement information from the sensor monitoring unit 200 to control a light source of the light irradiation unit 300.

For example, the controller 500 may be implemented by a processor, program instructions performed by the processor, software modules, microcode, computer program products, logic circuits, application-specific integrated circuits, firmware, and the like.

According to an example of the present disclosure, a fluid management unit 400 configured to supply the fluid to expand the balloon catheter 100 and to remove the supplied fluid may be included.

For example, the fluid management unit 400 may be controlled by the controller 500.

For example, the fluid injected into the balloon catheter 100 by the fluid management unit 400 is not particularly limited and may be a gas such as air or a liquid such as distilled water, saline solution, heavy water, and other contrast agents.

For example, the fluid management unit 400 may include an air trap removal unit 410 capable of removing an air trap inside the balloon catheter 100, and a cooler (not illustrated) configured to generate cooling fluid to lower the temperature of the fluid.

When the fluid injected by the fluid management unit 400 is a liquid, the air trap may be formed inside the balloon catheter 100 or in a fluid tube 420 which is a portion of the fluid management unit 400 and injects the fluid, and thus, the air trap removal unit 410 for removing the air trap may be included.

When performing a medical procedure using the air trap removal unit 410, side effects caused by air being trapped inside the balloon catheter 100 are minimized. Since the air trap inside the balloon catheter 100 may change the distribution of light irradiated from the optical fiber 320 in an uncontrollable manner, the air trap removal unit 410 may be provided to solve the problem.

Before injecting liquid into the balloon catheter 100, air bubbles representing the air trap may be sensed in advance and eliminated using IR light detectors or other technologies such as ultrasound imaging.

Additionally, the cooler prevents tissue damage by cooling the tissues, especially the mucosa or submucosa. For example, a cooling irrigation via a pump or expander may be included. The fluid flow and temperature within the balloon catheter 100 may be measured by the sensor 221, and the temperature and a flow rate may be transmitted to the monitoring device and may be controlled by the controller 500. Specifically, the controller 500 may control the supply, flow, or temperature of the fluid supplied and intaken from the fluid management unit 400. Additionally, the fluid management unit 400 may further include a fluid tube 420 configured to transmit the fluid.

For example, when the sensor 221 or the sensor array 220 is attached to a portion of the balloon catheter 100, the physical parameters of the tissue may be sensed. For example, the physical parameters may be tissue stress-strain, a pH level, and mucosal impedance of the mucosal surface. A treatment process may be monitored through these physical parameters. However, the sensing is not limited to the sensing of the physical parameters, and by recording electrical signals from a nervous system through single or multiple sensors 221, unwanted events occurring to the patient during treatment may also be sensed.

The sensor monitoring unit 200 may estimate the diameter of the balloon 150 from the monitored and measured pressure of the balloon 150 and select and control the optimal dosage, and when necessary, the sensor monitoring unit 200 may adjust the treatment dose according to the measured temperature and may control the target temperature to be maintain between 45 and 65 degrees, thereby protecting the tissue mucosa and submucosa from overheating. Additionally, a level of gastric acid may be defined according to several physical parameters, including the measured pH level, or the LES is near the heart by analyzing electrical signals coming from the esophageal tissue, the impedance of the mucous membrane and the nerves, which may assisting to control a heart rate as well as effects thereof on the nervous system during treatment, and for safety reasons, a mandatory control mode may be provided to terminate the treatment when temperature or strain stress exceeds a threshold value.

According to an embodiment of the present disclosure, an optical fiber 320, a sensor wire 210, a fluid tube 420, and an optical fiber moving unit 310 may be included. The sensor wire 210, the optical fiber 320, and the fluid tube 420 may be integrated and connected to an inner lumen of a delivery tubing 340.

The optical fiber moving unit 310 controls an movement of the optical fiber 320 inserted into the balloon catheter 100. Here, the optical fiber 320 inside the balloon 150 may be moved manually or automatically along a rail in the optical fiber moving unit 310 (i.e., translational or rotational movement), so that lesions of vascular tissue may be treated more broadly and selectively.

For example, the optical fiber moving unit 310 may include an optical fiber handling unit 311 including a translation controller 311a configured to control translational movement and a rotation controller 311b configured to control a rotational movement.

In this case, the optical fiber moving unit 310 may be used by including gears, wheels, and motors to control the movement of the optical fiber 320.

For example, a stopcock may be provided to be used as a connecting member configured to integrate fluid supply channels of the optical fiber 320, the sensor monitoring unit 200, the sensor wire 210 and the fluid management unit 400 into one pipe and insert the one pipe into the delivery tubing 340.

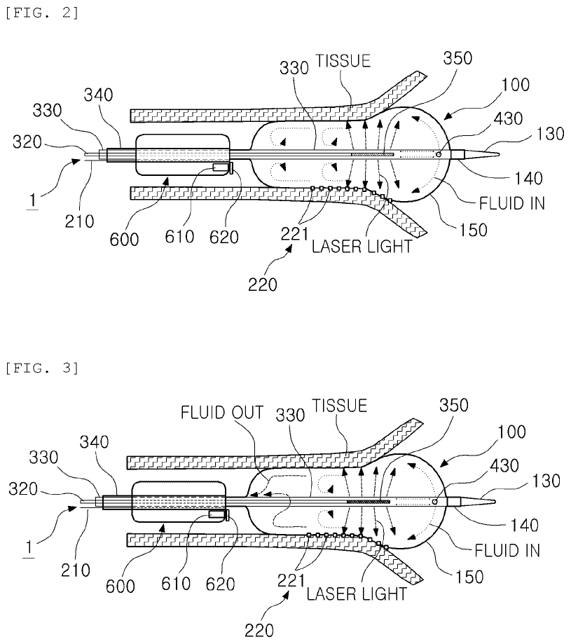

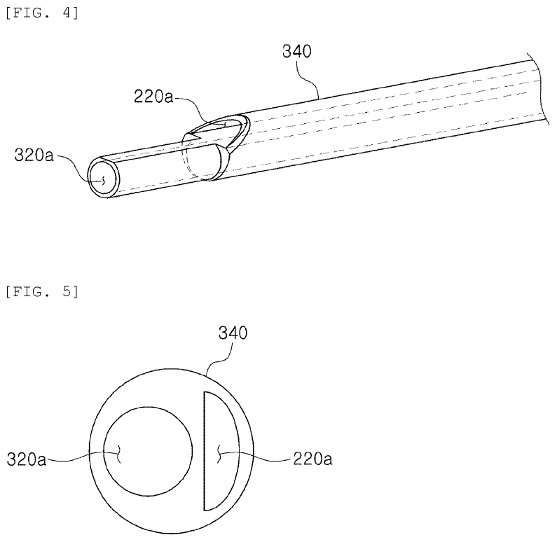

FIGS. 2 and 3 illustrate a state in which fluid is injected into an apparatus for laser treatment using a balloon catheter, and FIG. 2 illustrates a concept of fluid movement through unidirectional fluid injection, and FIG. 3 illustrates a concept of fluid movement through fluid injection and discharge.

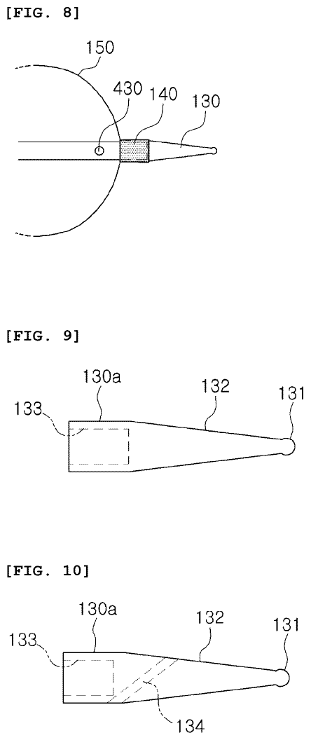

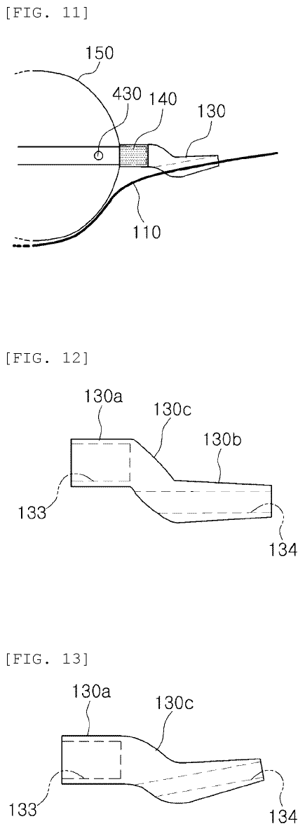

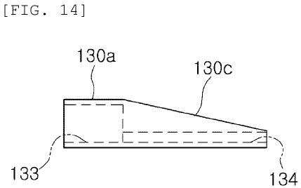

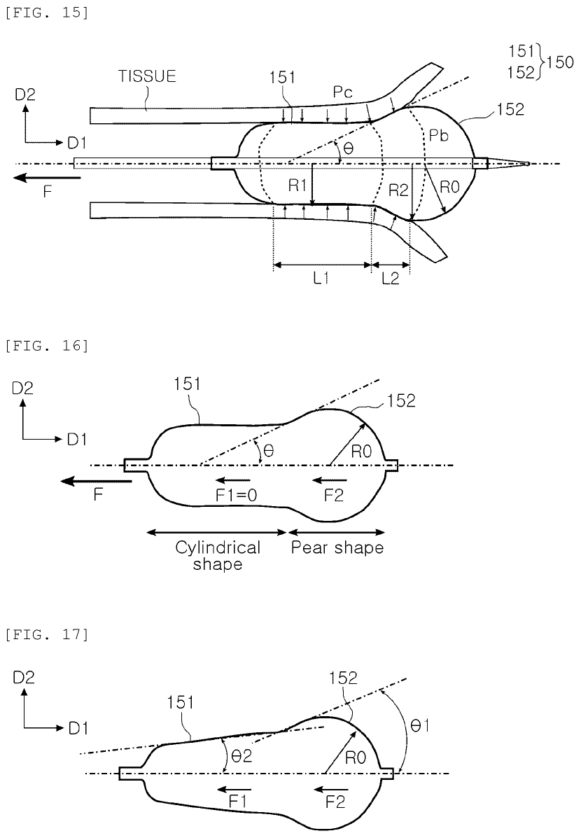

According to an example embodiment of the present disclosure, the balloon catheter 100 may include an optical fiber 320, an optical fiber tip 350, a balloon 150, a flexible tip 130, and a guide wire 110 (see FIG. 8).

The optical fiber 320 may be formed to allow light generated from a light source to travel therein. Additionally, an optical fiber tip 350 may be formed to surround the optical fiber 320 and to uniformly distribute the light emitted from the optical fiber 320 to the esophageal tissue. Additionally, the balloon 150 is formed of a transparent material so that the light emitted from the optical fiber tip 350 is irradiated to the esophageal tissue, and is expanded by the fluid flowing thereinto. The balloon 150 may include a first balloon portion 151 and a second balloon portion 152. Additionally, a flexible tip 130 disposed in a front end of the balloon catheter 100 is included for guiding and insertion into the esophageal tissue. The flexible tip 130 may be formed of a flexible material and prevent tissue damage when the balloon catheter 100 enters the tissue. Additionally, a guide wire 110 which is a wire configured to guide by penetrating through the flexible tip 130, may be included.

A plurality of sensors 221 may be provided on an outer side of the balloon catheter 100 to form a sensor array 220. Specifically, the plurality of sensors 221 may be provided outside the first balloon portion 151. Accordingly, it may be possible to monitor whether conditions set in a position of the first balloon portion 151 are satisfied with priority over the second balloon portion 152.

Additionally, a transparent tube 330 and a delivery tubing 340 are further provided inside the balloon catheter 100, so that the transparent tube 330 is disposed inside the delivery tubing 340 by penetrating through the delivery tubing 340, and the delivery tubing 340 may move the transparent tube 330. In the transparent tube 330, an optical fiber 320, a sensor wire 210 connected to the sensor array 220, and the like, may be disposed and moved integrally with the transparent tube 330.

For example, an endoscope unit 600 formed to surround at least a portion of the delivery tubing 340 which is disposed on the outside of the delivery tubing 340 and in a rear end in an entry direction of the balloon catheter 100 may be further provided.

The balloon catheter 100 is designed to be inserted into the endoscope channel in a through-the-scope (TTS) method, and an inner diameter of the insertion internal channel may be 2.8 to 4.3 mm. The optical fiber 320 is connected to a laser light source, so that the light is irradiated from the end of the optical fiber 320.

The optical fiber 320 may be comprised of a core, cladding, and a buffer for a multimode, and the core may have a thickness of 0.2 to 1 mm. An outer diameter of the optical fiber 320 is 0.3 to 1.5 mm depending on the purpose, and total length is designed according to an endoscope length and may be 1 to 4 m, but is not limited to this value.

The optical fiber tip 350 disposed in an end of the optical fiber 320 has an active length of 0.5 to 20 mm depending on the length of a target tissue and may irradiate the light. The optical fiber tip 350 may be cylindrical and may irradiate uniform light, and the light may pass through the transparent balloon 150 and reach the target tissue.

The optical fiber tip 350 may be protected with a highly transparent glass cap and may be sealed with high-melting point epoxy. Additionally, a material of the glass cap may include quartz, pure silica, polymethyl methacrylate (PMMA), and the like.

According to an example embodiment of the present disclosure, a filter 620 attached to the endoscope unit 600 and configured to protect the endoscope camera 610 may be included.

In order to protect the image sensor, such as CMOS, in the endoscope camera 610 and minimize light saturation, the endoscope camera 610 may be attached using the filter 620. This may assist to clearly visualize an entire procedure and a treatment area. There are several methods to attach the filter 620 to the camera 610, including attaching the filter 620 to a distal end of the endoscope using an epoxy adhesive or a redesigned silicone end cap, and the filter 620 is comprised of multiple layers and may include glass or thin, flexible plastic. Additionally, the filter 620 may be formed as a thin film having a thickness of 100 μm to 1 mm.

For example, the optical fiber tip 350 may move translationally and rotationally along the transparent tube 330. The transparent tube 330 may be disposed inside the balloon 150, and may be disposed in a center when a shape of the balloon 150 is symmetrical. However, when the balloon 150 is not symmetrical in shape, or is even symmetrical, a position of the transparent tube 330 may vary as needed.

When the transparent tube 330 is disposed in the center of the balloon 150, this may assist to transmit light energy to tubular tissue through cylindrical light distribution.

For example, the transparent tube 330 may be formed of acrylic, PET, polyethylene block amide, nylon, polyurethane, polycarbonate, and the like. Hardness may vary from Shore A to D. An inner diameter of the transparent tube 330 may be 1 to 3 mm, and a thickness thereof may be 0.2 to 1 mm to be sturdy, so that the optical fiber tip 350 may be configured so as not to be damaged even if optical fiber tip 350 is hit during moving.

For example, a cooling fluid may be injected to flow along an inner surface of the balloon 150 or a movement path of the optical fiber 320.

The cooling fluid or fluid for expansion may be injected into an interior of the balloon 150 through a fluid hole 430 formed in the transparent tube 330.

FIG. 2 illustrates a first embodiment in which the fluid is injected into the interior of the balloon catheter 100. A movement path of the fluid inside the balloon catheter 100 is indicated by a dotted curve, and a path along which light propagates is indicated by different arrows. As illustrated in FIG. 2, an interior of the balloon catheter 100 may be filled with the fluid so that the fluid may move along an inner surface of the balloon 150. Alternatively, the fluid may be injected to move along the outside of the transparent tube 330, that is, along the movement path of the optical fiber 320.

FIG. 3 illustrates a second embodiment in which the fluid is injected and discharged into the balloon catheter 100. When the fluid is injected into the balloon catheter 100, some of the fluid is injected along the inner surface of the balloon 150 as in the first embodiment and moves along the transparent tube 330, and another fluid may be discharged by moving along the inner surface of the balloon 150 and an outer edge of the transparent tube 330. This flow is indicated by a relatively thin line.

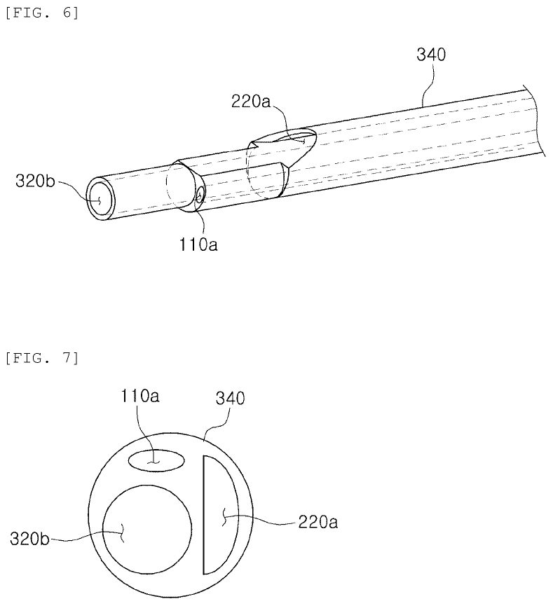

FIGS. 4 and 5 are views illustrating a transmission tubing having a dual channel. FIG. 4 is a perspective view, and FIG. 5 is a cross-sectional view.

A delivery tubing 340 may have two channels configured to support unidirectional injection of the fluid into a balloon 150. A first channel 220a for accommodating the sensor wire 210 may be connected to a sensor array 220. Additionally, the shape of a lumen of a second channel 320a relative to the position of the transparent tube in which an optical fiber 320 is accommodated and the balloon catheter 100 is expanded by fluid injection may be circular, semicircular, oval, or other shapes.

FIGS. 6 and 7 illustrate a transmission tubing having triple channels. FIG. 6 is a perspective view, and FIG. 7 is a cross-sectional view

The triple channel may be used for a fluid irrigation inside the balloon catheter 100 to assist to cool an inner surface of a transparent tube 330 and an optical fiber tip 350.

As described in FIG. 4, which is a dual channel, the first channel 220a accommodating a sensor wire 210 may be semicircular and may accommodate the optical fiber 320.

A third channel 110a may fill the balloon catheter 100 with the fluid and may be formed in a circular shape. Then, the transparent tube 330 may be disposed.

Unlike the second channel 320a of the dual channel, the second channel 320b of the triple channel may be used as a passage for cooling water to generate a cooling irrigation. The transparent tube 330 for accommodating both the optical fiber 320 and a coolant may be inserted into the second channel 320a, or may be covered on the outside of the second channel 320a.

The transparent tube 330 and the delivery tubing 340 may be coupled through an epoxy adhesive or other thermal bonding methods such as laser or electrical methods. A diameter of the delivery tubing 340 may be 1 to 4 mm depending on the inner diameter of the endoscope. The shape of the delivery tubing 340 may be various, such as circular, semicircular, or oval, and material included in the delivery tubing 340 may be polytetrafluoroethylene (PTFE), polyethylene, nylon (66, 11, 12), PET, and a block copolymer comprised of a rigid polyamide block and a flexible polyether block, and the like.

FIGS. 8,9,10,11,12,13 and 14 illustrate various example embodiments of a flexible tip. FIGS. 8,9 and 10 illustrate flexible tips in which an end is rounded. FIGS. 11,12,13 and 14 illustrate flexible tips having a step portion. FIGS. 8 and 11 illustrate a state in which the flexible tip is connected to a balloon, which illustrate a state including a portion of the balloon, and FIGS. 9 10,12,13 and 14 illustrate separate embodiments of the flexible tip.

FIGS. 8,9,10,11,12,135 and 14 illustrate that the flexible tip may be used when there is a guide wire 110, and specifically, FIGS. 10,11,12,13 and 14 illustrate a shape in which a passage 134 through which a guide wire 110 may penetrate is formed.

Referring to FIG. 9, the guide wire 110 is not inserted into the flexible tip 130 and may be disposed in an end of a balloon catheter 100, and the flexible tip 130 has a structure 132 that gradually becomes thinner toward a front end of the tip, and is formed in a round sphere shape 131 in the front end of the flexible tip 130, so that when the flexible tip 130 is in contact with the balloon 150, the balloon 150 is prevented from being broken, and may be inserted without causing mechanical damage to tissues inside the body. A groove 133 into which an optical fiber 320, and the like, may be inserted may be formed on one side thereof.

Referring to FIG. 10, a passage through which a guide wire 110, and the like, may be inserted may be formed.

FIGS. 11,12,13 and 14 illustrate a flexible tip 130 having the step portion formed, and FIG. 12 illustrates that a shape of the flexible tip 130 is comprised of an upper portion 130a inserted and fixed into a balloon 150 and a lower portion 130b through which the guide wire 110 penetrates, and the upper portion 130a and the lower portion 130b may be formed to have a step portion 130c. In this case, the flexible tip 130 may be formed in a shape in which the step portion 130c of the upper portion 130a and the lower portion 130b have a tapered shape.

Additionally, as illustrated in FIG. 13, the upper portion 130a and the lower portion 130b of the flexible tip 130 may be formed with the tapered step portion 130c, and the lower portion 130b may also be formed to have a shape tapered at a predetermined angle. That is, these portions may be formed as a tapered tip with a specific angle for inserting the guide wire 110, which may position the guide wire 110 near an axis of the balloon 150.

As illustrates in FIG. 14, two through-portions may be connected so that the guide wire 110 is inserted together with the tube and may be connected in series so that only the guide wire 110 may be further passed forward.

In this case as well, the tapered step portion 130c may be configured to be connected to the upper portion 130a.

Additionally, unlike FIGS. 8,9,10,11,12,13 and 14, the flexible tip 130 may be designed with an axis off center.

For example, the flexible tip 130 may be formed of a block copolymer comprised of a rigid polyamide block and a flexible polyether block, polyurethane, silicone, rubber, or other materials. Depending on the size and position of tubular tissue in the body, the flexible tip 130 may be designed to a desired size and configuration. In addition, radiopaque radio markers 140 may be used in proximal and distal portions of the balloon 150 to assist to identify an exact position of the balloon catheter 100 during treatment through X-ray images, and the radiopaque radio marker 140 may be formed of a highly reflective material, and also, in order to guide the balloon catheter 100 to a tubular sphincter tissue, the guide wire 110 may be inserted through the balloon 150 or out of the balloon. A material of the guide wire 110 may be a nitinol alloy or stainless steel, and the guide wire 110 may have a diameter of 0.021 to 0.038 inches and a length of 150 to 450 cm.

For example, the transparent tube 330 may include a hole formed for fixing the sensor array 220.

FIG. 15 illustrates a shape of a balloon according to a first embodiment. FIGS. 16 and 17 are auxiliary drawings illustrating the shape of a balloon according to the first embodiment. The shape of the balloon included in an apparatus for laser treatment using a balloon catheter having the balloon according to the first embodiment will be described below.

In the balloon 150 according to the first embodiment of the present disclosure, the first balloon portion 151 and the second balloon portion 152 may be expanded to a region rotated about a movement direction D1 of the optical fiber 320.

The first balloon portion 151 and the second balloon portion 152 may be connected to each other, and may be expanded to the region rotated around a moving path of the optical fiber 320, and may achieve a symmetrical shape.

As illustrated in the drawing, a length covered by the first balloon portion 151 in a direction D2 perpendicular to the movement path may be defined as a first radius R1. In this case, the first radius R1 may be 7.5 to 15 mm based on a 15 to 30 mm diameter standard of esophageal tissue. Additionally, when a length covered by the second balloon portion 152 in a direction perpendicular to the movement path may be defined as a second radius R2, the second radius R2 may be larger than the first radius R1 and smaller than 20 mm, which is a standard diameter of a cardia of the esophagus. Due to such a radius, maximum friction may be reduced, and the balloon catheter 100 may be disposed and fixed to minimize the maximum friction or respiratory problems due to the patient's movement during treatment. Additionally, light may be irradiated uniformly to tissues requiring the treatment.

FIG. 15 is a schematic diagram illustrating the balloon 150 in contact with a tubular sphincter tissue and a gastrocardiac tissue using the balloon catheter 100, and the shape of the balloon 150 may formed by rotating a straight line around one axis, for example, by rotating the first balloon portion 151, which is a cylindrical portion, and a curved line around one axis, and for example, a second balloon portion 152 having a pear shape may be coupled thereto. In this case, the shape of the second balloon portion 152 may assist to fix the balloon catheter 100 into the body tissue, and may provide greater coverage in treatment lumens of varying diameters, such as a gastroesophageal junction or a proximal edge of a hiatal hernia (gastrocardiac). In addition, the balloon 150 may act as an anchor configured to firmly fix a position in the tissue before treatment.

Specifically, the cylindrical shape is disposed in the esophageal tissue and sphincter tissue lesion areas so that the optical fiber 320 may easily cover these areas with light. For example, in consideration of the radius of the tissue as described above, a range of the first radius R1 may be 7.5 to 15 mm. The range may be a range to be safely disposed in the tissue such as the esophagus with a minimal movement without causing friction with the tissue. In addition, considering the patient's breathing and treatment, it may be possible to provide a constant distance for uniform laser irradiation from a center of an esophageal lumen to a treatment tissue on a surface of the esophagus.

The larger distal lumen disposed proximal to the hiatal hernia (gastrocardiac lesion) may result in a larger diameter in an end of the balloon 150.

The second balloon portion 152 may be disposed in a gastrocardiac area and may not only fixe the balloon catheter 100 through pulling force F, but also allow the treatment area to be expanded to the gastrocardiac area. A gastric cardia may be a junction between a lower esophageal sphincter and a stomach including a special group of muscles surrounding an esophageal junction. In this case, in order to treat the gastrocardiac lesion, the second balloon portion 152 may be designed in a pear-like shape so that the balloon catheter 100 may be fixed to the gastric cardia through contractile force and a treatment area may be expanded to the stomach.

For example, when a first balloon contact region in contact with the first balloon portion 151 is defined as A1, and a second balloon contact region in contact with the second balloon portion 152 is defined as A2, A1 and A2 may allow for contact with both a gastroesophageal junction and a proximal edge of a hiatal hernia, and a contact radius of tubular tissue may be defined as a radius of the esophageal tissue and a radius of the gastrocardiac area by the first radius R1 and the second radius R2, respectively, as illustrated in FIG. 15.

A contact length between the first balloon portion 151 and the tissue may be defined as L1, and a contact length between the second balloon portion 152 and the tissue may be defined as L2. In this case, θ, which is an angle between a tangent line in contact with the widest portion of the balloon 150 and a movement direction, and a total contact area, A, may be specified by the following equation.

$$A = {A}_{1} + {A}_{2} \\ {A}_{1} = 2 π {R}_{1} {L}_{1} \\ {A}_{2} = π ( {R}_{1} + {R}_{2} ) {L}_{2}$$

Here, R2 may be derived by the following equation.

$${R}_{2} = {R}_{1} + 2 {L}_{2} tan θ \\ {R}_{2} : {R}_{1} \to {R}_{0}$$

In the aforementioned equation, R0 refers to a radius of rotation of the second balloon portion 152 on a rotating body, and a contact radius is R2, which is derived from the range of R1 to R0.

For example, since a range of the first radius R1 is 7.5 to 15 mm, a range of the rotation radius R0 of the second balloon portion 152 may be 11 to 20 mm, and a range of the first length L1 may be 10 to 100 mm. To be further limited, the first length L1 may range from 20 to 50 mm to cover the gastroesophageal junction and a portion of the esophagus.

Additionally, the second length L2 is a range that may allow for complete contact with an entire gastric cardia based on a divergence angle of the gastric cardia, and may be in the range of 10 to 30 mm.

When the balloon 150 is in contact with the tissue, a contact pressure Pc is generated between the balloon 150 and the tissue. When a thickness of the balloon 150 is thin and the balloon 150 is flexible, a material leading to the balloon 150 has a minor effect on the tissue in terms of pressure change. Accordingly, the contact pressure Pc is essentially the same as the pressure Pb used to inflate the balloon 150, and in order to withdraw the inflated balloon catheter 100, axial force F must be applied to completely contact a distal end of the balloon 150 with a hiatal hernia. For example, assuming the esophageal tissue is a cylindrical tubular tissue and the frictional force applied to the tissue surface is minimal, the pulling axial force F may be defined as follows. As illustrated in FIGS. 16 and 17, force acting on a portion of the balloon 150 may be defined as F1 and F2, respectively, and resultant force may be axial force F. Hereinafter, the axial force F is as follows.

$$F = {F}_{1} + {F}_{2} \\ {F}_{1} = 0 \\ {F}_{2} = {A}_{2} {P}_{B} sin θ$$

However, there is a practical limit to the force F that may be applied to remove the balloon catheter 100 to prevent damage to the hiatal hernia. In order to measure the force F, the stress/strain may be recorded through the sensor array 220 and a signal may be transmitted to a controller 500 to sense critical force.

For example, an angle θ between any tangent line encountered in the second balloon portion 152 and the movement path may be 20 to 60°.

The balloon 150 must be connected to the hiatal hernias of various sizes. For example, the balloon 150 must have the second balloon portion 152, which is a large diameter distal end to fix the balloon 150 in a heart position. In this case, a range of 0, which is an angle between the tangent line in contact with the widest portion of the balloon 150 and the movement direction may be 20 to 60 degrees. Accordingly, between the first balloon portion 151 and the second balloon portion 152, the balloon portion 150 may be gently and evenly bent gradually to form a shape thereof compatible with a geometry of a junction between the LES and the gastrocardiac area. A conical taper angle (0) of the balloon 150 is in the range of 20 to 60 degrees, which may prevent the balloon 150 from moving backwardly into the esophageal tissue due to external force F.

For example, a material of the balloon 150 used may be urethane, silicone rubber, pellethane, and the like, with a hardness ranging from 90 to 50 Shore A, and may be inflated to a pressure of 0.1 to 5 PSI when in use.

For the used tissue, since a diameter of the esophagus ranges from 15 to 30 mm and a diameter of the hiatal hernia ranges from 22 to 40 mm, a range of the first radius R1 may be 7.5 to 15 mm and a range of the rotation radius R0 of the second balloon portion 152 may be 11 to 20 mm, so that the used tissue may be expanded to the same diameter range, and a contact between the balloon 150 and the tissue becomes larger, thereby providing an effect of covering a wide area without moving the balloon catheter 100.

Referring to FIG. 17, in the balloon 150 according to the first embodiment, the first balloon portion 151 may be formed in a straight line in a cross-section, and may be formed to have a second angle θ2 with the movement direction. In this case, the first angle θ1 may be derived with the same definition as the angle θ described above. Such a shape may vary depending on the design, and in this case, the second angle θ2 may always be smaller than the first degree 01.

For example, the first angle θ1 may be formed at 20 to 60°, and may provide maximum friction force in a fixed position. Due to 0 to 2 cm long hiatal collapse and the absence of a flap valve, the second angle θ2 for complete contact of the balloon with an esophageal surface may be 5 to 25°. The first balloon portion 151 and the second balloon portion 152 may gradually merge with each other because a change in angle is not extremely large.

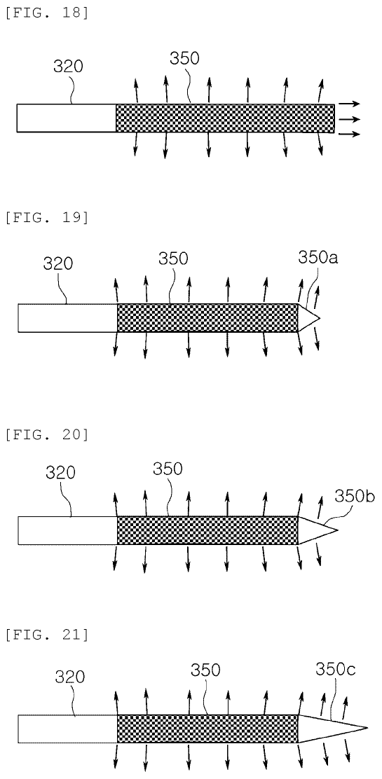

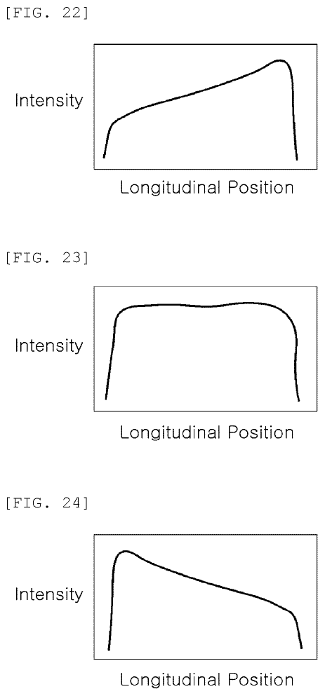

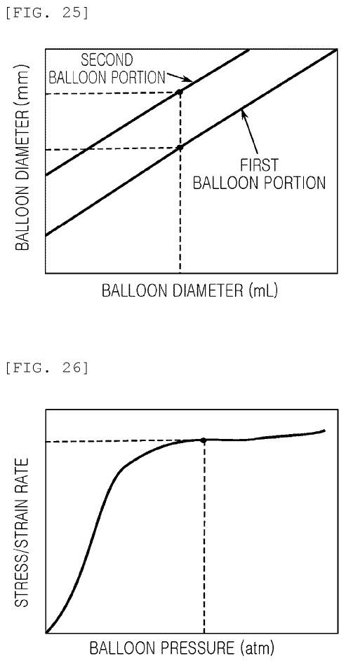

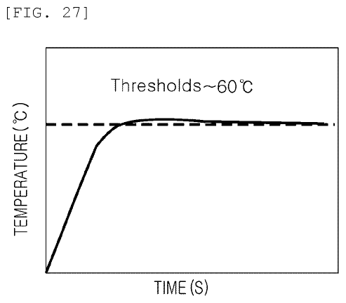

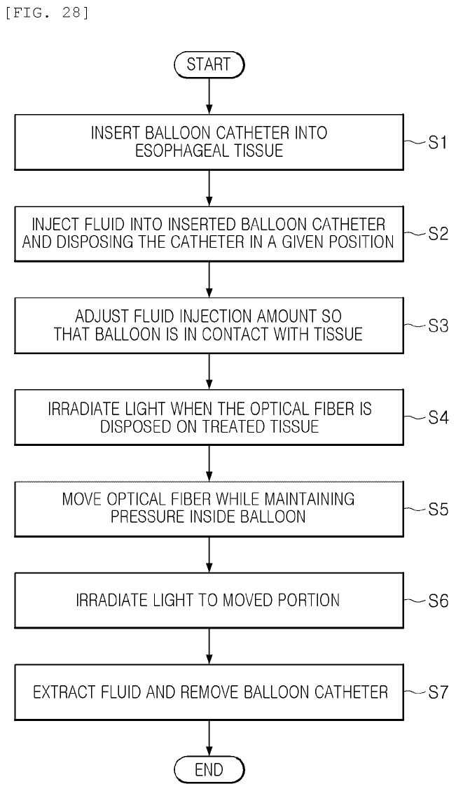

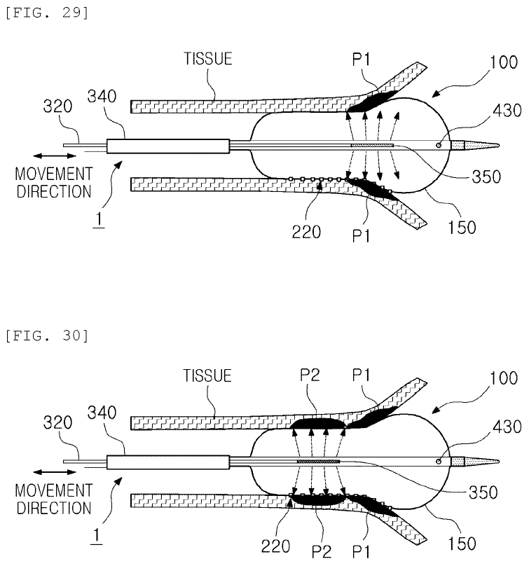

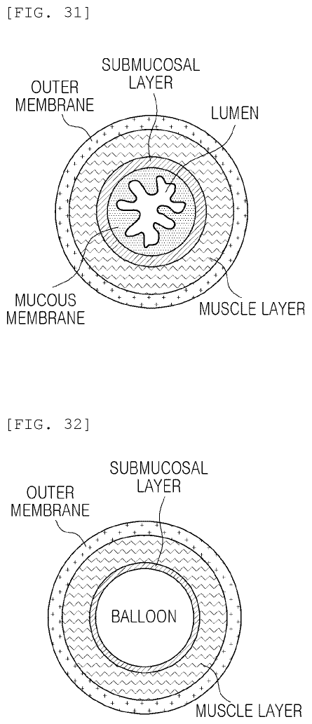

FIG. 18 illustrates a first embodiment of an optical fiber tip connected to an optical fiber, and FIGS. 19,20 and 21 illustrate a case in which a conical diffusion tip for diffusion is further formed on an optical fiber tip. FIGS. 22,23 and 24 are graphs illustrating an amount of light intensity change according to a position when the optical fiber tips of FIGS. 19,20 and 21 are used, and FIG. 19 is illustrated as FIG. 22, FIG. 20 is illustrated as FIG. 23, and FIG. 21 is illustrated as FIG. 24.

Referring to FIG. 18, when the optical fiber tip 350 is not provided with a diffusion tip, light leakage occurs from an end of the optical fiber tip 350.

As illustrated in FIGS. 19,20 and 21, when the optical fiber tip 350 is provided with the diffusion tip, a path of light output by the diffusion tip may be changed, and thus the light output from an end thereof may be used.

FIGS. 1920 and 21 illustrate that shapes of diffusing tips 350a, 350b and 350c are different from each other, and the diffusing tips 350a, 350b and 350c may emit light in radial emission according to the conical shape, angle, and roughness of the surface. Here, a cone angle of the diffusion tips 350a, 350b and 350c is a decisive factor in achieving radially uniform light emission and minimum front light emission, and it may be effective to set the cone angle in the range of 5 to 90°.

In case of having a cone angle of 25° in FIG. 20, while a relatively symmetrical spatial light distribution is generating along an axis of the optical fiber 320, as illustrated in FIG. 23, another angle in FIG. 19 illustrates a light distribution tilted to the right as in FIG. 22 (i.e., a larger cone angle equal to or greater than 40°), and when the light distribution is slightly biased to the left in FIG. 21 (i.e., smaller cone angle below 10°), the light distribution is slightly biased to the right as in FIG. 24. Since the intensity thereof varies depending on the cone angle at each position, the intensity may be selectively applied when irradiating light to a specific disease state.

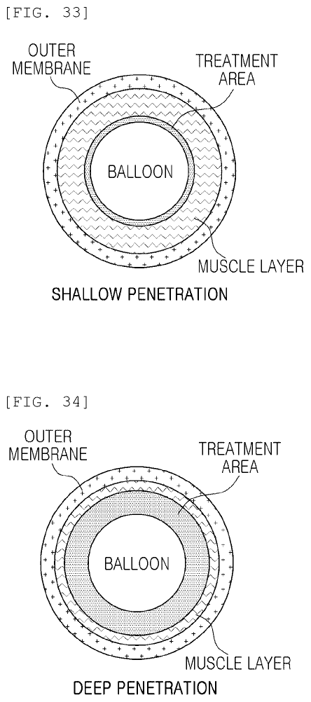

FIG. 25 illustrates a relationship between a balloon diameter and a fluid injection amount, FIG. 26 illustrates a relationship between stress/strain rate and balloon pressure Pb, and FIG. 27 is a graph illustrating a temperature change during treatment over time.

FIG. 25 represents a fluid injection amount in terms of volume and illustrates diameters of the first balloon portion 151 and the second balloon portion 152 accordingly. Accordingly, it may be found that the diameters of the first balloon portion 151 and the second balloon portion 152 increase depending on the amount of injected fluid, and it may be found that the diameter of the second balloon portion 152 is always larger than the diameter of the first balloon portion 151. That is, in order to improve a contact surface between the tissue and the balloon 150 and completely adhere the balloon 150 to the tissue, pressure of the balloon 150 is measured and a diameter of the balloon 150 is estimated. Additionally, the amount of fluid injected may be selected accordingly an appropriate treatment amount.

For example, the range of the injection amount is 15 to 50 ml, and the first radius R1 may be expanded from 7.5 mm to 15 mm, and the second radius R2 may be expanded from 22 mm to 40 mm.

FIG. 26 is a graph illustrating pressure of the balloon 150 according to the stress/strain rate of the balloon 150, and illustrates an occurrence of the pressure Pb of the balloon 150 and the local deformation (stress) due to the expansion of the balloon 150 and illustrates a relationship between the stress/strain rate and the pressure of the balloon 150 based on a signal of the stress/strain rate measured by the sensor 221. The stress (force/area; N/m²) is deformation force due to the pressure of the balloon 150 acting on a unit area of the esophageal tissue, and the deformation of the tissue due to this stress is referred to as a strain rate (difference ΔL between the deformed length and the original length/original length L0; mm), which is a value obtained by measuring the deformation of the esophageal tissue compared to the original shape of the tissue. When liquid is injected into the balloon 150, not only the diameter of the balloon 150 increases, but also the pressure of the balloon 150 increases, but the stress generated therein acts on the tissue, which may cause the deformation of the tissue and a decrease in mucosal/submucosal thickness (ΔL/L0; mm) as compared to the original tissue. The relationship between the pressure Pb of the balloon and tissue contact pressure Pc is illustrated in FIG. 15. For example, due to a thin and flexible balloon 150, when the balloon is in complete contact with the tissue, the tissue contact pressure Pc is equal to the pressure Pb of the balloon. Additionally, a signal of the stress/strain may not only confirm a diameter of the balloon 150, but also record undesirable effects such as breakage of the balloon 150 or leakage of the balloon 150.

FIG. 27 is a graph illustrating a temperature change measured by a sensor 221 to maintain a temperature. That is, the sensor 221 may provide feedback to the controller 500 to adjust a treatment amount and set a target temperature to 45 to 65° C. to prevent the mucosa and submucosa from overheating. The temperature and stress/strain of a surface of the treated tissue or the balloon 150 have threshold values, and the controller 500 may control both the temperature and the pressure not to be exceeded. Additionally, the sensor array 220 may be attached to the surface of a tissue-balloon 150 to sense not only a tissue temperature, but also tissue deformation, a PH level and mucosal impedance, and even electrical signals of the nervous system, and the sensor array 220 may include a thermocouple sensor 221, a fiber Bragg gating (FBG) sensor 221, and an optical fiber 320, thereby allowing the apparatus for laser treatment using the balloon catheter 100 to be accurately controlled to avoid tissue damage.

For a treatment method according to the following embodiments, the contents described above will be described without any change.

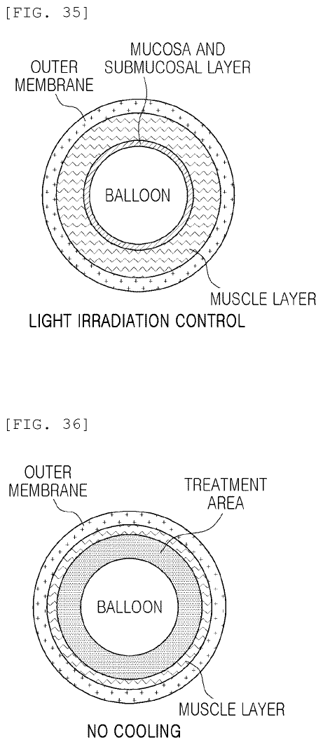

FIG. 28 is a flowchart of a treatment method using an apparatus for laser treatment using a balloon catheter according to an example embodiment of the present disclosure.

For example, an insertion operation (S1) of inserting the balloon catheter 100 through an endoscope channel, an inflation operation S2 of injecting fluid into the balloon catheter 100 and disposing the balloon catheter in a esophageal tissue, an adjustment operation (S3) of adjusting the amount of the fluid to closely adhere to an irradiation position through the sensor 221 provided on an outer side of the balloon catheter 100, and an irradiation operation of irradiating light to the irradiation position and a movement operation of moving the position of the optical fiber 320 inside the balloon catheter 100 and irradiating light (S5 and S6). Additionally, when the irradiation is completed and the treatment is completed, a removal operation (S7) of removing the balloon catheter 100 after extracting the fluid to make the balloon 150 smaller may be further included.

In the insertion operation (S1), a guide wire 110 may be used to assist in insertion of the catheter.

Then, in the operation, the balloon 150 is inflated by injecting the fluid until the removal operation (S7), but since an air trap removal unit 410 is provided, side effects due to the generation of air bubbles may be minimized. Here, the air trap may be sensed with an IR light detector or other techniques may be used to sense air bubbles inside the fluid tube 420.

The balloon catheter 100 may be adjusted so that the balloon 150 is disposed in the treatment position, and a circular marking line may be marked around the surface of the balloon 150, so that the balloon catheter 100 may be easily disposed in a desired location. Depending on patient parameters, the amount of fluid may be further increased to ensure complete contact between the surface of the balloon 150 and the tissue, and for example, the fluid management unit 400 may fill the fluid at a desired pressure within the range of 0.1 to 5 psi. Additionally, a treatment area is changed as the optical fiber 320 moves through the transparent tube 330 inside the expanded balloon catheter 100. A radio marker 140 may be attached to ends of the optical fiber 320 and the balloon 150 to accurately confirm the position in which light is transmitted. The position of the balloon catheter 100 and the treatment position may be confirmed by X-ray imaging before the medical procedure.

For example, in order to treat the gastrocardiac lesion, the laser is first delivered to the hiatal hernia lesion, and then, while maintaining the pressure of the balloon 150, the optical fiber 320 is removed from a sphincter position for a secondary treatment. Depending on the length of the treatment area, the doctor may treat various lesions by moving the optical fiber 320 inside the balloon 150 without shrinking the balloon 150. Since the balloon catheter 100 may be inserted into an endoscope, the entire process (before, during, and after treatment) may be visualized through the endoscope, and for example, an endoscope camera 610 may be a camera 610 connected to a monitor and a PC.

Since the lesions may be treated in the same manner as above, even when treatment of tissues in a plurality of positions is required, the treatment may be performed by moving the position of the optical fiber 320 inside without changing the position of the balloon 150. Accordingly, the risk of damaging tissue may be reduced, and the treatment time may be reduced because the balloon 150 itself does not move.

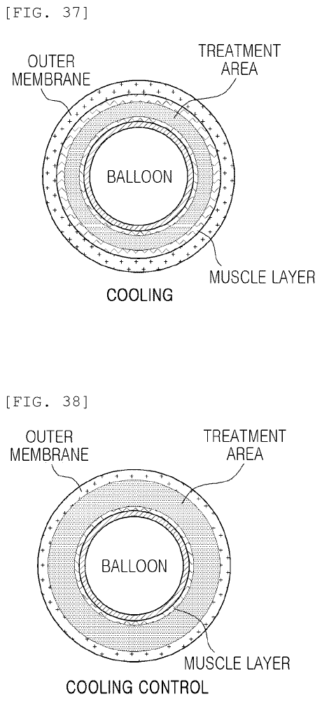

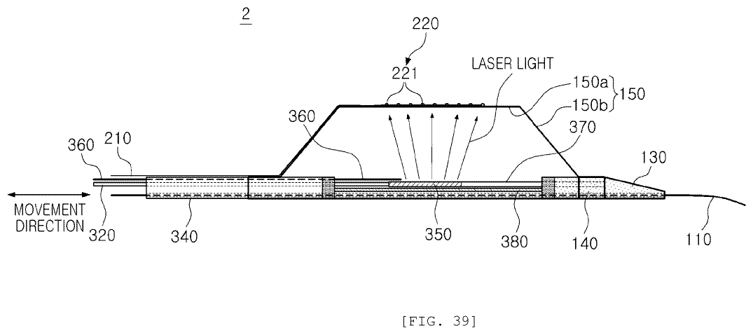

FIGS. 29 and 30 illustrate treating a plurality of treatment areas without moving the position of the balloon catheter inside the human body. FIG. 29 illustrates primary treatment of a first area P1, and FIG. 30 illustrates secondary treatment of a second area P2.

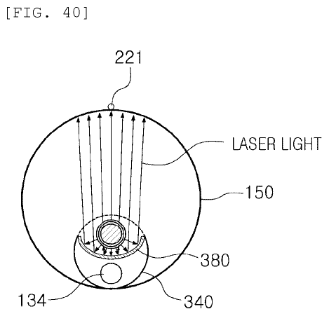

FIGS. 31,32,33 and 34 are a cross-sectional view of tubular tissue after laser light treatment according to the present disclosure. As illustrated in FIG. 31, FIG. 31 illustrates a typical tubular tissue which shows a flexible structure with several layers such as mucosa, a submucosal layer, a muscle layer, and adventitia. FIG. 32 illustrates that a balloon 150 is inflated after a balloon catheter 100 reaches the target lesion. In this case, it may be found that thicknesses of the mucosa and the submucosal layer is significantly reduced to 1 to 3 mm and a uniform layer is formed around a surface of the balloon 150. Additionally, as illustrated in FIGS. 33 and 34, light irradiation may select a wavelength depending on a heat penetration depth of a tissue target layer. That is, for heat treatment of a shallow layer of the tissue in FIG. 33, examples of applicable wavelengths include 405, 490, 532, 585, 755, 980, 1470, 1550 and 2200 nm, and the treatment area may produce ablation, removal, destruction, and/or solidification thickness of 0.5 to 2 mm. In this case, a radiation exposure range may be 0.1 to 1 kJ/cm2 and an output range may be 5 to 100 W.

Additionally, examples of wavelengths applicable for heat treatment of deep tissue layers in FIG. 34 may include 630, 808, 980, 1064, and 1300 nm, and the treatment area produces a solidification thickness of 2 to 5 mm. In this case, the radiation exposure range may be 0.01 to 1 J/cm2 and the output range may be 100 mW to 50W.

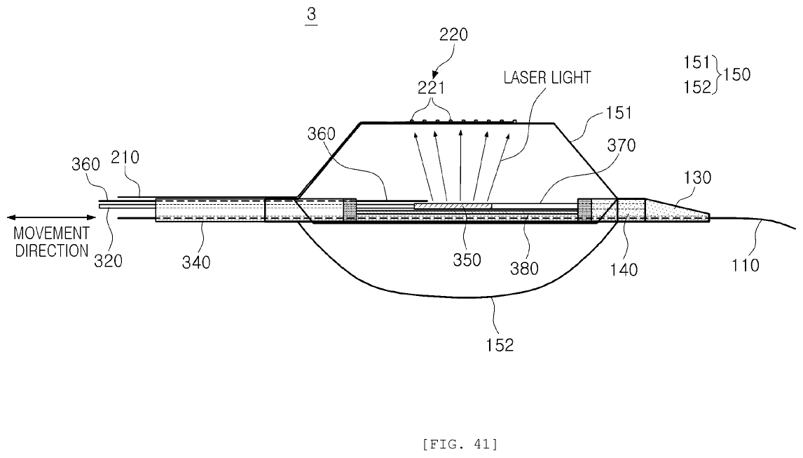

FIGS. 35,36 and 37 are cross-sectional views of tubular tissue illustrating an effect after a cooling process during laser light treatment according to the present disclosure. As illustrated in FIGS. 35,36 and 37, if there is no cooling process after treatment due to light irradiation in FIGS. 35 and 36, thermal damage may occur in mucosa and a submucosal layer, which may cause side effects during laser treatment.

Before irradiating the light, an operation of cooling a surface of a treatment tissue by introducing fluid into the balloon catheter 100 may be performed, and more specifically, the fluid management unit 400 serves to cool the mucosa and the submucosal layer to protect the tissue during photothermal treatment with laser light after injection of the fluid. That is, the fluid management unit 400 supplies the fluid to the inflated balloon 150 by a pump. In this case, the flow and temperature of the fluid in the balloon catheter 100 may be controlled to an appropriate temperature and flow by the controller 500 based on data sensed by the sensor monitoring unit 200. Here, the fluid may include various components such as distilled water, saline solution, heavy water, and a contrast agent, and may be supplied to the balloon 150 through an expander or a pump. If necessary, an initial temperature of the fluid may be set to 5 to 30° C. before supplying the fluid. As illustrated in FIG. 37, when the cooling process is performed, a degree of coagulation lesions may be controlled during laser treatment. The cooling process may protect 1 to 2 mm thick mucosal surfaces from potential excessive heat during treatment using the cooling fluid at 4 to 10° C. That is, in order to maintain the treatment temperature at a desired temperature (45 to 65° C.) by the controller 500, as illustrated in FIG. 38, the temperature of the sensed mucosal surface is recorded and feedback is transmitted to the controller 500. Accordingly, a mucosa-submucosa protection is performed by cooling liquid, a coagulation thickness is controlled, and a target temperature is maintained using the controller 500. Here, the data monitored by the sensor array 220 may be transmitted to the controller 500 to obtain a balloon pressure level on the tissue surface, stress strain, electrical signals of nerves and heartbeat, and a temperature of the tissue surface.

Embodiments according to the shape of the various balloons 150 described below will refer to the above-described purpose, structure and effect, unless contrary to the above-mentioned content.

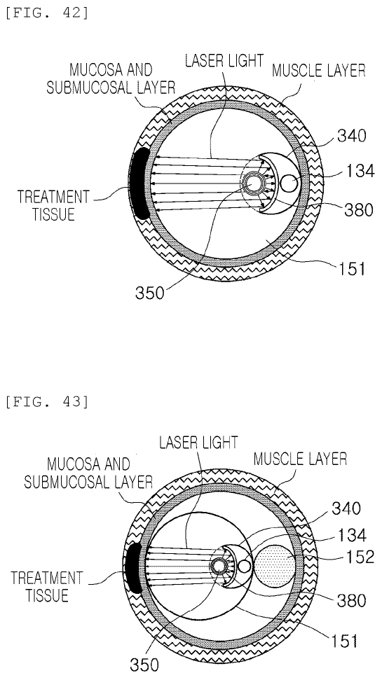

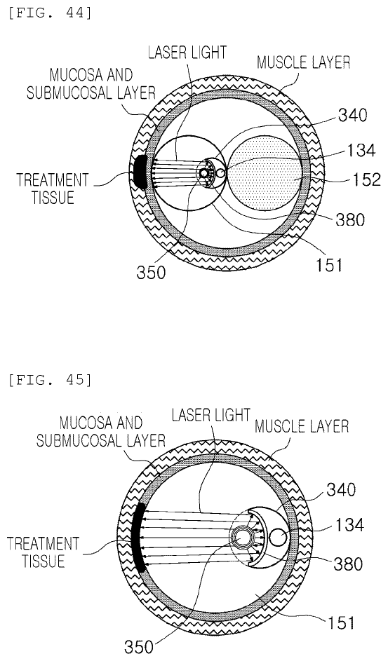

FIGS. 39 and 40 show apparatus for laser treatment using a balloon catheter having a shape of a balloon according to a second embodiment. FIGS. 39 and 40 are cross-sectional views illustrating cross-sections, perpendicular to each other.

The apparatus 2 for a laser treatment using a balloon catheter having a balloon according to a second embodiment according to an example embodiment of the present disclosure may include a light irradiation unit 300 configured to irradiate light to esophageal tissue, a balloon catheter 100 into which the light irradiation unit 300 is inserted, and which is inflated by fluid so that the light irradiation unit 300 is disposed on one side of an interior thereof and expands an internal tissue of the esophagus, a sensor monitoring unit 200 configured to monitor the esophageal tissue sensed by the sensor 221 provided in the balloon catheter 100, and a controller 500 configured to receive measurement information from the sensor monitoring unit 200 to control a light source of the light irradiation unit 300.

For example, the balloon 150 may include a straight portion 150a in parallel with a movement direction, and a connection portion 150b connected to the straight portion 150a and the delivery tubing 340. The optical fiber 320 connected to the light irradiation unit 300 may be expanded by the fluid to be disposed on one side of an interior of the balloon 150, and may be formed to be contact with the balloon 150 due to the pressure expanded by the fluid only in a portion of the esophageal tissue.

According to an example embodiment of the present disclosure, an optical mask 360 may be further included.

The optical mask 360 may be configured to block irradiation of light generated from the optical fiber tip 350 and prevent tissues that do not need irradiation of laser light from being affected.

In an example, the optical mask 360 is disposed over the fiber tip 350 to limit an active length for treatment lesion selection, and the optical mask 360 may be formed of an opaque chromium metal layer on a glass substrate. Additionally, the optical mask 360 may be translated and moved along the optical tip.

Additionally, by providing the optical mask 360, a position of the balloon catheter 100 may be accurately identified during treatment by X-ray imaging using a wireless radio marker 140 in an end of the balloon 150.

According to an example embodiment of the present disclosure, a reflecting mirror 380 may be further included. The reflecting mirror 380 may reflect the light generated from the light irradiation unit 300 and may be disposed inside the balloon catheter 100.

Since light is irradiated from the optical fiber tip 350 in all directions, the reflecting mirror 380 may be further included inside the delivery tubing 340 so that the laser light may be appropriately irradiated to the treatment tissue. The reflecting mirror 380 is disposed on an opposite side of the treatment tissue relative to the optical fiber 320 and may change the light distribution from cylindrical emission to cross-sectional emission. For example, the reflectivity of high-power laser may be improved by forming the reflective mirror 280 by coating various materials such as copper, silver, aluminum, and gold in multiple layers, and the reflecting mirror 380 may be formed in a parabolic shape to irradiate the treatment tissue.

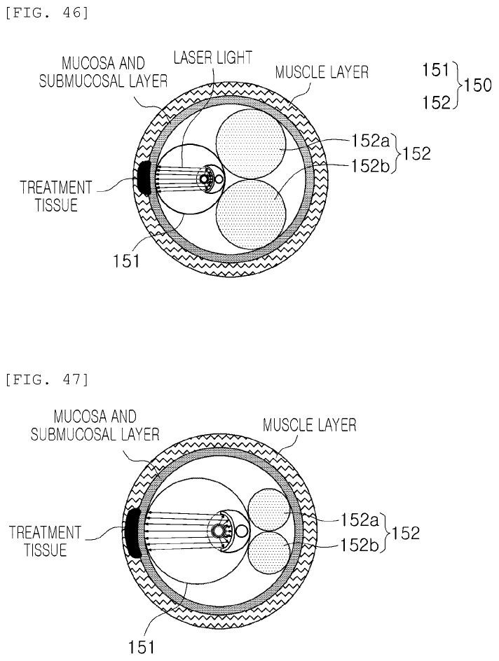

FIG. 41 shows an apparatus for laser treatment using a balloon catheter having a balloon according to a third embodiment of the present disclosure.

According to an example embodiment of the present disclosure, an apparatus 3 for a light treatment using a balloon catheter having a balloon according to the third embodiment may include a balloon catheter 100 including a first balloon portion 151 into which the optical fiber 320 is inserted and which is expanded by the fluid to expand the esophageal tissue and expands to form a straight line having an angle of 0 to 90° with a movement path of an optical fiber 320, and a second balloon portion 152 configured to expand in a curved line.

In this case, the first balloon portion 151 and the second balloon portion 152 may be disposed to overlap at least a partial region in the movement direction of the optical fiber 320. Additionally, the first balloon portion 151 and the second balloon portion 152 may be sealed with each other so that the fluid may be separately injected and expanded.

For example, the first balloon portion 151 may be mainly in close contact with the tissue to irradiate light, and the second balloon portion 152 may be a support balloon 150 configured to support the first balloon portion 151 so that the treatment may be performed smoothly.

For example, a plurality of second balloon portions 152 may be provided to stably support the first balloon portion 151. Since the second balloon portion 152 forms a curved line, an area in close contact with the tissue may be relatively smaller than that of the first balloon portion 151. Additionally, the first balloon portion 151 may be disposed stably by providing the plurality of second balloon portions 152.

FIGS. 42,43,44 and 45 schematically show the use of an apparatus for laser treatment using a balloon catheter with a balloon of a second embodiment or a third embodiment.

For example, in an apparatus 3 for a laser treatment using a balloon catheter having a balloon according to the third embodiment, a first balloon portion 151 and a second balloon portion 152 may be separately controlled by a controller 500.

FIG. 42 illustrates an apparatus 2 for a laser treatment using a balloon catheter having a balloon according to the second embodiment, where partial removal or thermal coagulation may occur due to side irradiation of the balloon catheter 100. In other words, the balloon catheter 100 may selectively treat target tissue while preserving surrounding healthy tissue.

FIGS. 43 and 44 illustrate that the diameter of the balloon 150 may be changed by adjusting the amount of fluid injected into the second balloon portion 152.

Depending on patient parameters, the diameter of the second balloon portion 152 may vary from 5 to 30 mm, and during the treatment process, the first balloon portion 151 may maintain a constant size of the balloon 150, that is, treatment results may be consistent with the same laser irradiation dose for all sizes of tubular tissue. In this case, a cooling liquid inside the balloon 150 may maintain the mucosa and the submucosal layer at a thickness of 1 to 2 mm. Additionally, FIG. 45 illustrates the removal or thermal coagulation changes in shallow tissue layers such as mucosa or submucosa layers using a wavelength band with a shallow penetration depth.

When a liquid at room temperature (20 to 25° C.), body temperature (37° C.), or high temperature (50 to 60° C.) is used instead of the cooling liquid used in of FIG. 42, thermal coagulation may be strengthened to remove even relatively deep tissue layers.

FIGS. 46,47 and 18 schematically illustrate the use of an apparatus for laser treatment using a balloon catheter having a balloon according to the third embodiment, and in this case, FIGS. 46,47 and 48 illustrate a case of having two second balloon portions.

The two second balloon portions 152a and 152b may improve a stability position of the balloon catheter 100, especially the active balloon 150 of the tubular tissue, and as illustrated in FIGS. 46 and 47, the two support balloons 150 may have the same size. Alternatively, the two support balloons 150 may have different sizes as in FIG. 48. Accordingly, the balloon catheter 100 may not only change overall dimensions but also adjust an angle (5) of a laser beam. Referring to FIG. 48, by inflating the balloon 150 again and then deflating the balloon 150 again, the balloon catheter 100 may change a treatment angle without reconfiguring the position of the balloon 150.

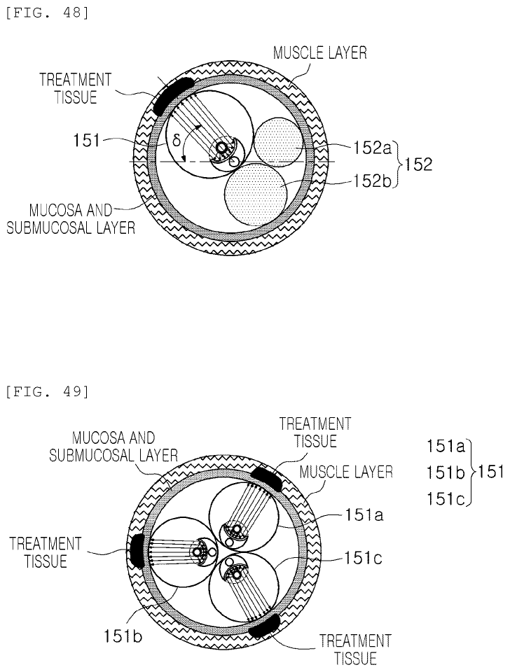

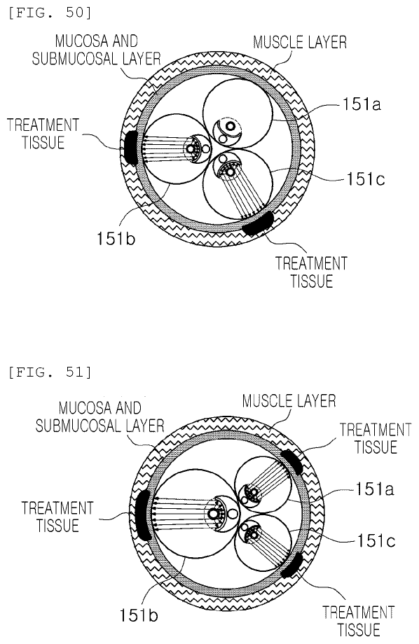

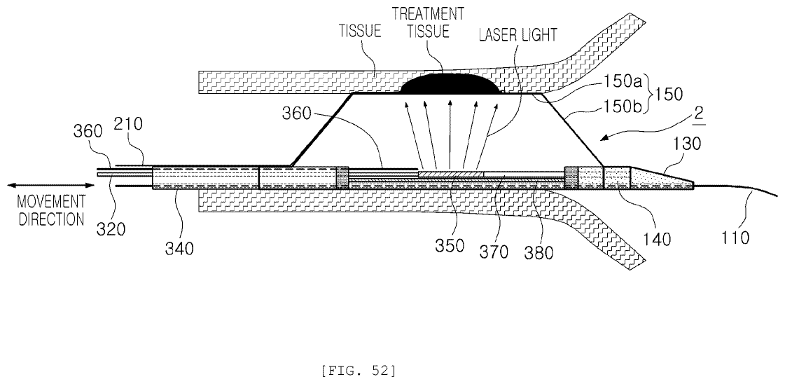

FIGS. 49,50 and 51 illustrate a case in which an apparatus for laser treatment using a balloon catheter with a balloon according to a second embodiment is provided with a plurality of balloon catheters.

According to an example embodiment of the present disclosure, the light irradiation unit 300 and the balloon catheter 100 may be provided in plural, and the controller 500 may individually control the light irradiation unit 300 and the balloon catheter 100.

An alternative design of an off-center axis of the balloon catheter 100 having the balloon 150 of the second embodiment may be comprised of three balloons 151a, 151b and 151c that may independently expand a diameter of the balloon 150 by irradiating laser light.

For example, FIGS. 49,50 and 51 illustrate cross-sectional views of tubular tissue after laser light treatment for the three balloons 151a, 151b and 151c, and the balloon catheter 100 in FIG. 20B illustrates two partial removals or thermal coagulation generated by two balloons 151b and 151c. In this manner, a size of the balloon 150 and the amount of laser radiation may be adjusted according to the size, area, treatment depth, and location of the target tissue, and accurate treatment may be provided through individual control.

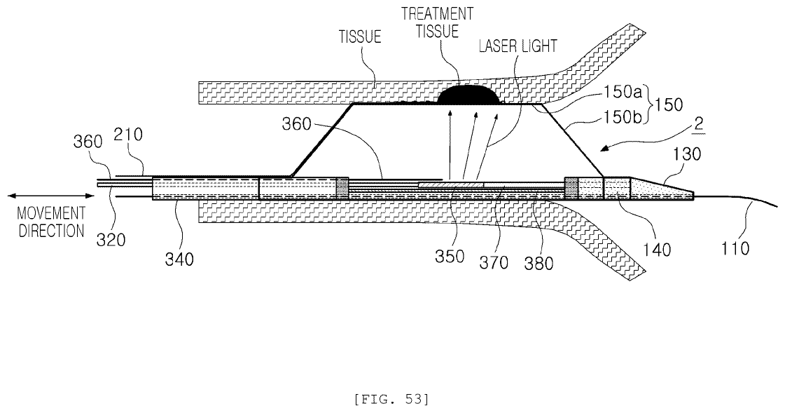

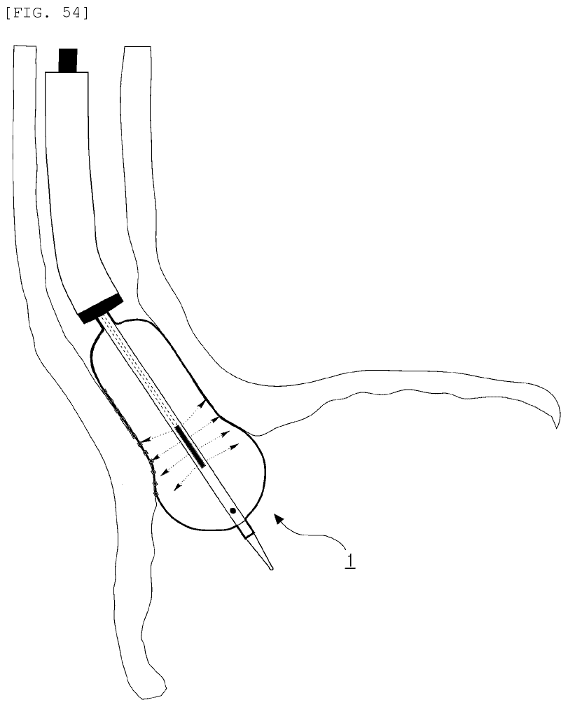

FIGS. 52 and 53 illustrate controlling an irradiation range of laser light by an optical mask in a balloon catheter having a balloon according to a second embodiment or a third embodiment.

As compared to FIG. 52, FIG. 53 illustrates that a range of the optical mask 360 covering the optical fiber tip 350 widens and a range of a treatment tissue narrows. The range may be adjusted as described above by moving the optical mask or moving an optical fiber 320.

As an example, the optical mask 360 may be translated and moved along the optical fiber tip 350 regardless of the movement of the optical fiber 320, and a wire of the optical mask 360 may move in a delivery tubing 340 and a proximal end of the optical mask 360 may be connected to a translation motor. A shape of a light beam is a rectangular metal plane with a width of 1 to 5 mm, a thickness of 0.1 to 1 mm, and a length of 5 to 40 mm, and may partially or completely cover the optical fiber tip 350. The optical mask 360 may adjust an active length from a diffusion tip to allow a treatment length to be adjusted without reducing a diffusion length, thereby providing an effect of providing more options to select treatment methods depending on the size of the treatment tissue. FIG. 52 illustrates that the optical mask 360 is disposed behind the optical fiber tip 350 to transmit all laser light to the tissue, and according to FIG. 21B, the optical mask 360 limits the transmission of the laser light irradiated from the optical fiber 320, so that the removal or solidification area is smaller than those of FIG. 52.

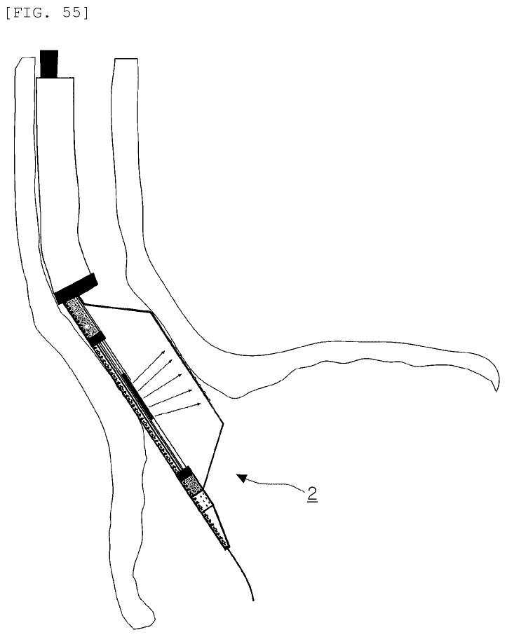

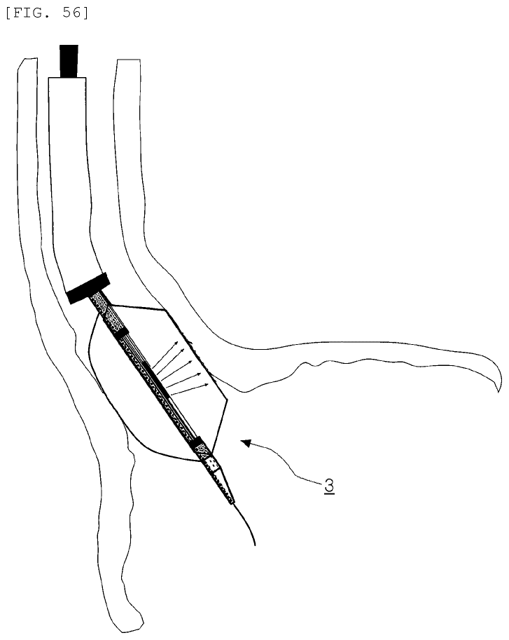

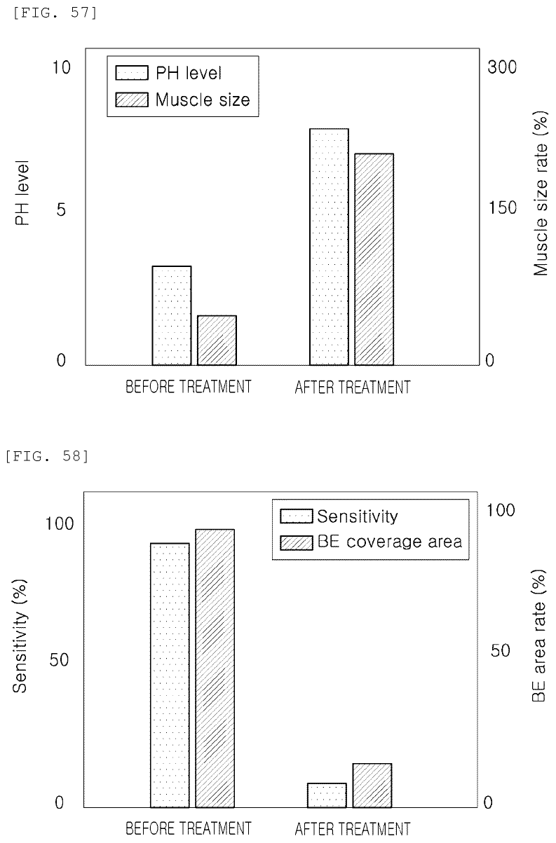

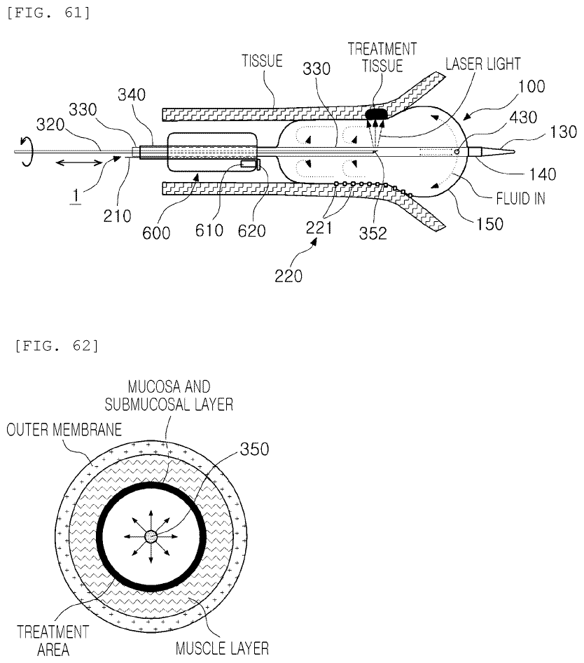

FIGS. 54 to 56 schematically illustrate a state in which an apparatus for laser treatment using a balloon catheter having a balloon of the first to third embodiments according to an example embodiment of the present disclosure is disposed in the tissue. FIG. 54 illustrates a balloon in the first embodiment, FIG. 55 illustrates a balloon in the second embodiment, and FIG. 56 illustrates a balloon in the third embodiment. FIGS. 27 and 58 illustrate graphs illustrating changes before and after treatment of treated gastro-esophageal reflux disease through an apparatus or method for a laser treatment using a balloon catheter according to an example embodiment of the present disclosure.

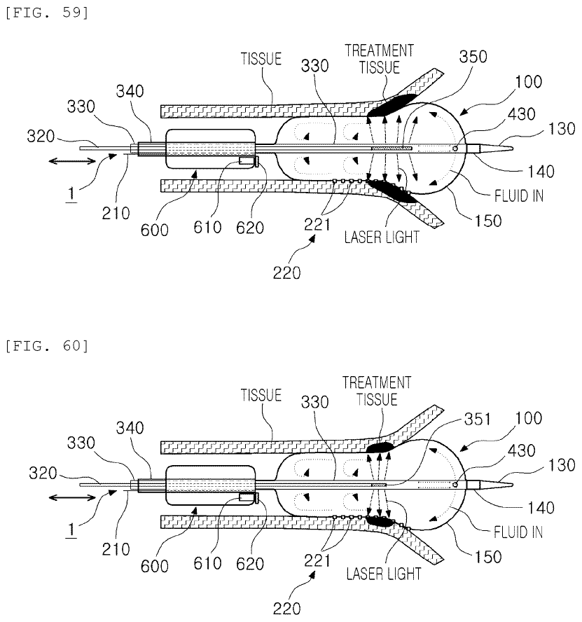

FIG. 57 graphically illustrates a change in esophageal acidity and an effect on muscle before and after treatment of gastro-esophageal reflux disease.

Stomach acid refluxes into the esophagus because a lower esophageal sphincter (LES) is weak or relaxed, and if not treated, some complications such as esophageal ulcer, esophageal stricture, and esophageal erosion may occur. Applying phototherapy to the LES and heart locations may provide improvements to strengthen the LES or repair hiatal hernias. The balloon catheter 100 may directly transmit the laser energy of a diffusion type applicator to the LES and a gastric cardiac portion for treatment, a target temperature of an esophageal muscularis propria may be increased to 65 to 85° C. using the controller 500 and the light irradiation unit 300, and light may be absorbed with a cooling liquid to maintain a mucous membrane temperature below 50° C., thereby performing treatment. Partial tissue coagulation in the LES and gastric cardiac locations is induced for the purpose of improving function. Ablation and coagulation of muscle tissue may stimulate wound healing, resulting in the growth of new collagen and elastin fibers, and these fibers tighten and thicken the LES muscle, which may assist to reduce reflux of stomach contents into the esophagus. Additionally, in esophageal reflux disease, changes in acidity in the esophagus before and after treatment are often monitored to evaluate the severity of acid reflux, and resection/coagulation of this tissue may assist to prevent acid reflux due to tightening and thickening of the LES muscle, thereby ensuring minimal or no reflux of stomach contents into the esophagus.

Accordingly, as illustrated in FIG. 57, an increase in a thickness of smooth muscle marked by muscle may assist sphincter muscle relaxation and flexibility in opening and closing, thereby providing the effect of alleviating the above problems. As a result, the pH level increases and reaches a normal pH in the esophagus (i.e., pH 7). It indicates that no or minimal acidic stomach contents are flowing back into the esophagus.

FIG. 58 is a graph illustrating a change in sensitivity and effective range of an electronic sensor through endoscopic screening before and after treatment of Barrett's esophagus.

Barrett's esophagus is a state in which a chronic complication of esophageal reflux disease may occur in an esophagus, where a mucous membrane of a distal portion of the esophagus connected to the stomach is exposed to stomach acid for a long time due to continuous reflux to change the esophageal mucosal tissue into a gastric mucosal tissue, that is, a state in which the mucous membrane inside the esophagus becomes thick and red. Such a change indicates a precancerous mucosal change and is associated with the development of esophageal adenocarcinoma, a type of cancer. Accordingly, when Barrett's esophagus phototherapy is used, an abnormal tissue inside the esophagus may be removed or cut, thereby reducing the risk of progression to esophageal adenocarcinoma.

The balloon catheter 1 illustrated in FIG. 54 treats Barrett's tissue by directly delivering the light energy of the diffusion applicator to the Barrett's tissue, and the temperature of the mucosa/submucosal esophageal tissue is set and maintained at 65 to 85° C. through sensor monitoring using the controller 500 and the light irradiation unit 300. The optical applicator may be moved inside the balloon to cover the entire Barrett's tissue through the optical fiber moving unit 310. Additionally, the balloon catheter 2 according to the second embodiment of FIG. 55 and the balloon catheter 3 according to the third embodiment of FIG. 56 may be applied to locally treat irregularly shaped Barrett's tissue. The entire procedure may be monitored in real time by an endoscope camera 610.

Barrett's tissue is red in color, and because of the shallow optical penetration and strong surface light absorption, the apparatus for laser treatment of the balloon catheter may selectively coagulate the mucosa or the submucosa, which is an area 1 to 2 mm below the mucosa, and cause no or minimal thermal damage to the muscle layer. The esophageal mucosa removed after treatment is replaced with normal esophageal mucosa through a regenerative process through an inflammatory response. In addition, measurement of changes in sensitivity of electronic sensors and endoscopy are major methods for diagnosing and confirming the treatment effect of BE. Accordingly, as illustrated in FIG. 58, regeneration of new normal tissue to replace the cut Barrett's tissue may be confirmed to have a decrease in sensitivity using an electronic sensor configured to sense biomarkers appearing in respiration, and the removal of the Barrett's tissue may be confirmed by confirming the effective range of light treatment through the endoscope.

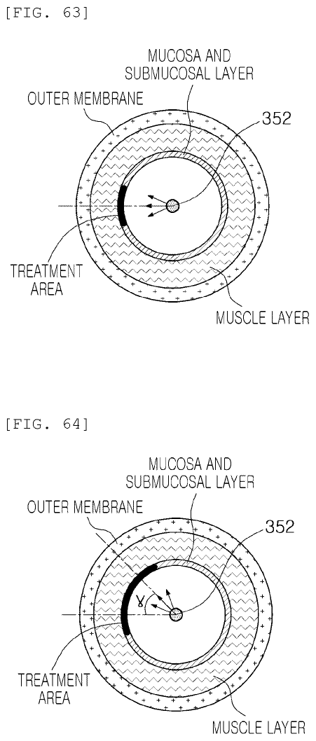

FIGS. 59,60 and 61 illustrate various shapes of optical fibers that may be included in the present disclosure, and FIG. 59 illustrates a light diffusing fiber, FIG. 60 illustrates a circumferentially diverging optical fiber, and FIG. 61 illustrates a side emitting optical fiber. FIG. 62 schematically illustrates a cross-section of the optical fiber in a state of light irradiation according to a shape of an optical fiber illustrated in FIG. 59 or 60, and FIG. 63 or 64 schematically illustrate a cross-section of the optical fiber in a state of light irradiation according to the shape of the optical fiber illustrated in FIG. 26C.

Referring to FIG. 59 and FIG. 62, when a light diffusion fiber 350 is provided, light may be generally emitted from a tip of the optical fiber along the circumferential direction and may have a relatively long treatment range. The treatment range may range from 10 to 30 mm.

Referring to FIG. 60 and FIG. 62, when a circumferentially diverging optical fiber 351 is provided, light may be generally emitted from the tip of the optical fiber along the circumferential direction, and the treatment range may be relatively short, in the range of 5 to 7 mm.

Referring to FIG. 61 and FIGS. 63 and 64, when a side-emitting optical fiber 352 is provided, light may be emitted to one side for treatment of local areas, and the—emitting optical fiber 352 may be provided to have a set angle γ in the circumferential direction. This angle γ may be adjusted. Additionally, a local treatment lesion area may be broadly set manually or automatically by rotating the side-emitting optical fiber 352, but a combination of translational and rotational movements may be used to enable localized to large-area treatments, which may improve the flexibility of the medical procedure.

As described above, there are various types of optical fibers, and depending on the design, any one or a plurality of optical fibers necessary for constructing the apparatuses 1, 2 and 3 for a laser treatment using the balloon catheter of the first to third embodiments may be selected.

As described above, the present disclosure has been described focusing on the example embodiments, but the present disclosure is not limited to the above-described example embodiments, and the example embodiments may be modified and implemented by those skilled in the art without changing the technical concept of the present disclosure as claimed in the claims.

What is claimed is:

1. An apparatus for laser treatment of gastro-esophageal reflux disease using a balloon catheter, comprising:
a light irradiation unit configured to irradiate light to esophageal tissue;
a balloon catheter including a first balloon portion into which an optical fiber connected to the light irradiation unit is inserted, and which is expanded by fluid to expand the esophageal tissue and is expanded to form a straight line having an angle of 0 to 90° with a movement path of the optical fiber, and a second balloon portion expanded to form a curved line;
a sensor monitoring unit configured to monitor esophageal tissue sensed by a sensor provided in the balloon catheter; and
a controller configured to receive measurement information from the sensor monitoring unit to control a light source of the light irradiation unit.

2. The apparatus for laser treatment of gastro-esophageal reflux disease using a balloon catheter according to claim 1, further comprising:
a fluid management unit configured to supply fluid to expand the balloon catheter and remove the supplied fluid,
wherein the fluid management unit is controlled by the controller.

3. The apparatus for laser treatment of gastro-esophageal reflux disease using a balloon catheter according to claim 1, wherein the first balloon portion and the second balloon portion are expanded into a region rotated around a movement path of the light irradiation unit.

4. The apparatus for laser treatment of gastro-esophageal reflux disease using a balloon catheter according to claim 1, wherein the first balloon portion and the second balloon portion are disposed to overlap each other in at least a partial region in a movement direction of the optical fiber.

5. The apparatus for laser treatment of gastro-esophageal reflux disease using a balloon catheter according to claim 4, wherein the first balloon portion and the second balloon portion are supplied with fluid separately by the controller.

6. The apparatus for laser treatment of gastro-esophageal reflux disease using a balloon catheter according to claim 1, wherein a plurality of the sensors form an array and are provided on an outer side of the first balloon portion.

7. The apparatus for laser treatment of gastro-esophageal reflux disease using a balloon catheter according to claim 2, wherein the fluid management unit includes an air trap remover configured to remove an air trap inside the balloon catheter, and a cooler configured to generate a cooling fluid for lowering a temperature of the fluid.

8. The apparatus for laser treatment of gastro-esophageal reflux disease using a balloon catheter according to claim 1, wherein the balloon catheter includes:
the optical fiber;
an optical fiber tip formed to surround the inserted optical fiber and configured to uniformly distribute light emitted from the optical fiber to the esophageal tissue;
a balloon formed of a transparent material so that light emitted from the optical fiber tip is irradiated to the esophageal tissue and expanded when fluid flows therein, and including the first balloon portion and the second balloon portion;
a flexible tip disposed in a front end to be guided and inserted into the esophageal tissue; and
a guide wire configured to guide by penetrating through the flexible tip.

9. The apparatus for laser treatment of gastro-esophageal reflux disease using a balloon catheter according to claim 8, wherein the sensor array measures a temperature, tissue deformation, PH, and a mucosal impedance of the esophageal tissue.

10. The apparatus for laser treatment of gastro-esophageal reflux disease using a balloon catheter according to claim 8, further comprising:
a delivery tubing provided with a first channel for accommodating a sensor wire connected to the sensor, a second channel for accommodating the optical fiber and allowing for entry and exit of a material to expand the balloon catheter, and a third channel for accommodating the guide wire to secure an entry path; and
an endoscope portion formed to surround at least a portion of the delivery tubing.

11. The apparatus for laser treatment of gastro-esophageal reflux disease using a balloon catheter according to claim 10, wherein the endoscope portion includes:
an endoscope camera; and
a filter attached to the endoscope portion and configured to protect the endoscope camera.

12. The apparatus for laser treatment of gastro-esophageal reflux disease using a balloon catheter according to claim 7, wherein the cooling fluid is injected to flow along an inner surface of the balloon or the movement path.

13. The apparatus for laser treatment of gastro-esophageal reflux disease using a balloon catheter according to claim 3, wherein when a length covered by the first balloon portion in a direction perpendicular to the movement path is defined as a first radius, the first radius is 7.5 to 15 mm, and when a length covered by the second balloon portion in the direction perpendicular to the movement path is defined as a second diameter, the second diameter is greater than the first radius and less than 40 mm.

14. The apparatus for laser treatment of gastro-esophageal reflux disease using a balloon catheter according to claim 3, wherein an angle between any tangent line in contact with the second balloon portion and the movement path is 20 to 60°.

15. The apparatus for laser treatment of gastro-esophageal reflux disease using a balloon catheter according to claim 4, further comprising:
a reflecting mirror configured to reflect light generated from the light irradiation unit and disposed inside the balloon catheter.

16. The apparatus for laser treatment of gastro-esophageal reflux disease using a balloon catheter according to claim 4, wherein the second balloon portion is provided in plural.

17. An apparatus for laser treatment of gastro-esophageal reflux disease using a balloon catheter, comprising:
a light irradiation unit configured to irradiate light to esophageal tissue;
a balloon catheter having the light irradiation unit inserted thereinto, expanded by fluid so that the light irradiation unit is disposed on one side of an interior, and configured to expand the esophageal tissue;
a sensor monitoring unit configured to monitor the esophageal tissue sensed by a sensor provided in the balloon catheter; and
a controller configured to receive measurement information from the sensor monitoring unit to control a light source of the light irradiation unit.

18. The apparatus for laser treatment of gastro-esophageal reflux disease using a balloon catheter according to claim 17, wherein the light irradiation unit and the balloon catheter are provided in plural, and
the controller individually controls the light irradiation unit and the balloon catheter.

19. The apparatus for laser treatment of gastro-esophageal reflux disease using a balloon catheter according to claim 17, provided with an optical mask configured to cover light emitted from an optical fiber tip disposed in an end of the optical fiber.

20. A method for a laser treatment of gastro-esophageal reflux disease using a balloon catheter, the method comprising:
an insertion operation of inserting a balloon catheter through an endoscope channel;
an inflating operation of injecting fluid into the balloon catheter and disposing the fluid on esophageal tissue;
an adjustment operation of adjusting an amount of fluid to be adjusted to an irradiation position through a sensor provided on an outer side of the balloon catheter;
an irradiation operation of irradiating light to the irradiation position; and
a moving operation of moving a position of an optical fiber inside the balloon catheter and irradiating light.
